(19) United States
(12) Patent Application Publication
(10) Pub. No.: US 2010/0130469 A1
(43) Pub. Date: May 27, 2010

(54) 2, 6-NAPHTHRIDINE DERIVATIVES

(76) Inventors: Maurice van Eis, St. Louis (FR); Walter Schuler, Grenzach-Wyhlen (DE); Anette Von Matt, Biel-Benken (CH); Nicolas Soldermann, Rosenau (FR); Lauren G. Monovich, Belmont, MA (US); Christoph Gaul, Aesch (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/594,724

(22) PCT Filed: Apr. 4, 2008
(86) PCT No.: PCT/EP2008/054104

§ 371 Date: Oct. 5, 2009

Related U.S. Application Data
(60) Provisional application No. 60/910,469, filed on Apr. 6, 2007.

(51) Int. Cl. A61K 31/397; A61K 31/551; A61K 31/499; A61K 31/496; A61K 31/4375; A61K 31/4725; C07D 471/04; C07D 401/14; C07D 217/12; C07D 205/04; A61P 37/00; A61P 29/00

(52) U.S. Cl. 514/210.21; 514/218; 514/253.04; 514/253.05; 514/300; 514/310; 540/575; 544/231; 544/362; 544/363; 546/122; 546/143; 548/953

(57) ABSTRACT

The present invention relates to novel organic compounds comprising a naphthyridine which may be mediators of a selective subset of kinases belonging to the AGC kinase family, such as for example PKC, PKD, PKN-1/2, CDK-9, MRCK-beat, PASK, PRKX, ROCK-I/II or mediators of other kinases, the selectivity of which would be depending on the structural variation thereof.

The present invention relates to organic compounds of the structural type shown below, which may be mediators of a selective subset of kinases belonging to the AGC kinase family, such as for example PKC, PKD, PKN-1/2, CDK-9, MRCK-beta, PASK, PRKX, ROCK-I/II or mediators of other kinases, the selectivity of which would be depending on the structural variation thereof.

In one aspect the present invention provides a compound of formula I

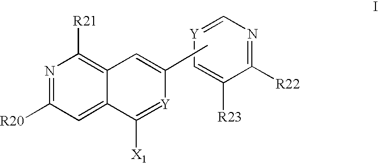

wherein X₁ is a ligand of formula (a), (b), (c), (d), or (e),

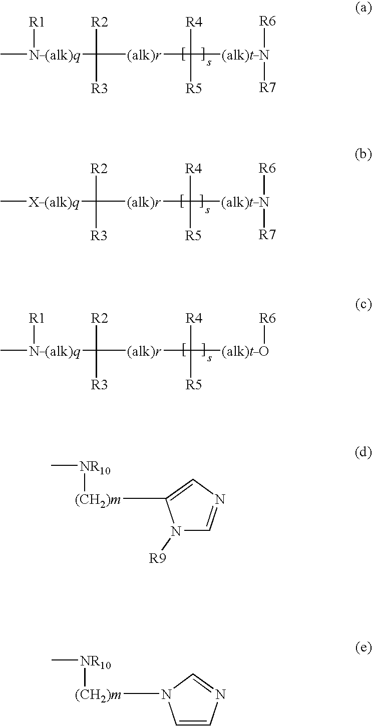

and wherein
X stands for O or S; preferably O,
alk stands for alkylene,
Y and Y₁ are independent from each other and stand for CH or N,
R₂₀ and R₂₁ are independently selected from the group consisting of hydrogen, cyano, amino, N-alkylamino, N,N-dialkylamino, —NH-alkylene-aryl, —NH-aryl, halo, alkoxy, hydroxyl, and mercapto;
R₂₂ is hydrogen,
R₂₃ is selected from hydrogen, lower alkyl, halo, hydroxyl, SH, CN and CF₃,
R₁ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl; or R₁ and R₂ are collectively alkyl and form together with the atoms to which they are attached a 4 to 8 membered ring system which may be interrupted by —O—, —S—, —NR₈—, or —CO—, or R₁ and R₄ are collectively alkyl and form together with the atoms to which they are attached a 4 to 8 membered ring system which may be interrupted by —O—, —S—, —NR₈—, or —CO—, or R₁ and R₆ are collectively alkyl and form together with the atoms to which they are attached a 4 to 8 membered ring system which may be interrupted by —O—, —S—, —NR₈—, or —CO—;
R₂ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl; or R₂ and R₃ are collectively alkyl and form together with the atoms to which they are attached a 3 to 7 membered ring system which may be interrupted by —O—, —S—, —NR₈—, or —CO—, or R₂ and R₄ are collectively alkyl and form together with the atoms to which they are attached a 5 to 10 membered ring system which may be interrupted by —O—, —S—, —NR₈—, or —CO—, or R₂ and R₆ are collectively alkyl and form together with the atoms to which they are attached a 5 to 10 membered ring system which may be interrupted by —O—, —S—, —NR₈—, or —CO—;
R₃ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl, or R₃ and R₁ are collectively alkyl and form together with the atoms to which they are attached a 5 to 10 membered ring system which may be interrupted by —O—, —S—, —NR₈—, or —CO—, or R₃ and R₄ are collectively alkyl and form together with the atoms to which they are attached a 5 to 10 membered ring system which may be interrupted by —O—, —S—, —NR₈—, or —CO—, or R₃ and R₆ are collectively alkyl and form together with the atoms to which they are attached a 5 to 10 membered ring system which may be interrupted by —O—, —S—, —NR₈—, or —CO—, or R₃ and R₂ combine together to one oxygen atom of a carbonyl-group;
R₄ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl; or R₄ and R₅ are collectively alkyl and form together with the atoms to which they are attached a 3 to 7 membered ring system which may be interrupted by —O—, —S—, —NR₈—, or —CO—, or R₄ and R₆ are collectively alkyl and form together with the atoms to which they are attached a 4 to 8 membered ring system which may be interrupted by —O—, —S—, —NR₈—, or —CO—, or R₄ and R₅ combine together to one oxygen atom of a carbonyl-group;
R₅ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl; or R₅ and R₆ are collectively alkyl and form together with the atoms to which they are attached a 4 to 8 membered ring system which may be interrupted by —O—, —S—, —NR₈—, or —CO—, or R₅ and R₃ are collectively alkyl and form together with the atoms to which they are attached a 4 to 8 membered ring system which may be interrupted by —O—, —S—, —NR₈—, or —CO—, or R₅ and R₆ are collectively alkyl and form together with the atoms to which they are attached a 4 to 8 membered ring system which may be interrupted by —O—, —S—, —NR₈—, or —CO—;
R₆ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl; or R₆ and R₇ are collectively alkyl and form together with the atoms to which they are attached a 3 to 8 membered ring system which may be interrupted by —O—, —S—, —NR₈—, or —CO—;
R₇ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl; or R₇ and R₅ are collectively alkyl and form together with the atoms to which they are attached a 5 to 10 membered ring system which may be interrupted by —O—, —S—, —NR₈—, or —CO—, or R₇ and R₃ are collectively alkyl and form together with the atoms to which they are attached a 5 to 10 membered ring system which may be interrupted by —O—, —S—, —NR₈—, or —CO—;
R₉ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl; preferably hydrogen or alkyl, more preferably hydrogen or lower alkyl,
R₁₀ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl; preferably hydrogen or alkyl, more preferably hydrogen or lower alkyl,
m is an integer and is from 1-8,
q, r, s, and t are independent from each other and stand for 0 or 1.

In the above definitions wherein two variables can form together a 3 or higher membered ring system it is preferred that a 3 membered ring system is not interrupted by either —O—, —S—, —NR₈—, or —CO—.

### Other Preferences

Preferably, Y stands for N.

For Y₁═N, the pyrimidin ring in formula (I) may be a 2-, 4- or 5-pyrimidyl substituent, preferably a 4-pyrimidyl substituent in accordance to formula (IIa).

For Y₁═CH, the pyridyl substituent in formula (I) may be a 2-, 3, or 4-pyridyl substituent, more preferably a 4-pyridyl substituent in accordance to formula (IIb).

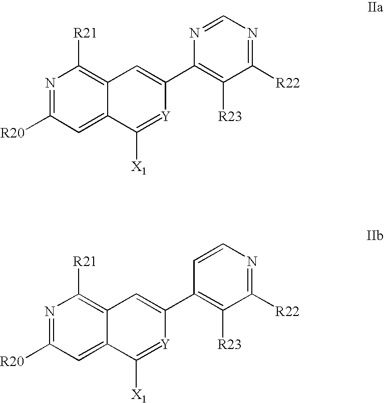

Y in a compound of formula IIa or IIb is preferably N.

A compound in accordance to formula IIb wherein Y═N is more preferred, has formula III and the variables are as defined herein:

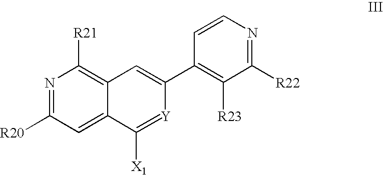

The ligand X₁ is preferably selected from a residue in accordance to formula (a), (b) and (c), more preferably from formula (a) and (b), even more preferably ligand X₁ is of formula (a).

Preferably the variables R₁ through R₇ are:

R₁ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl; or R₁ and R₂ are collectively alkyl and form together with the atoms to which they are attached a 4 to 8 membered ring system, or
R₁ and R₄ are collectively alkyl and form together with the atoms to which they are attached a 4 to 8 membered ring system, or R₁ and R₆ are collectively alkyl and form together with the atoms to which they are attached a 4 to 8 membered ring system;
R₂ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl; or R₂ and R₃ are collectively alkyl and form together with the atoms to which they are attached a 3 to 7 membered ring system, or R₂ and R₄ are collectively alkyl and form together with the atoms to which they are attached a 5 to 10 membered ring system, or R₂ and R₆ are collectively alkyl and form together with the atoms to which they are attached a 5 to 10 membered ring system;
R₃ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl, or R₃ and R₄ are collectively alkyl and form together with the atoms to which they are attached a 5 to 10 membered ring system, or R₃ and R₄ are collectively alkyl and form together with the atoms to which they are attached a 5 to 10 membered ring system, or R₃ and R₆ are collectively alkyl and form together with the atoms to which they are attached a 5 to 10 membered ring system, or R₃ and R₂ combine together to one oxygen atom of a carbonyl-group;
R₄ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl; or R₄ and R₅ are collectively alkyl and form together with the atoms to which they are attached a 3 to 7 membered ring system, or R₄ and R₆ are collectively alkyl and form together with the atoms to which they are attached a 4 to 8 membered ring system, or R₄ and R₅ combine together to one oxygen atom of a carbonyl-group;
R₅ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl; or R₅ and R₆ are collectively alkyl and form together with the atoms to which they are attached a 4 to 8 membered ring system, or R₅ and R₃ are collectively alkyl and form together with the atoms to which they are attached a 4 to 8 membered ring system, or R₅ and R₆ are collectively alkyl and form together with the atoms to which they are attached a 4 to 8 membered ring system;
R₆ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl; or R₆ and R₇ are collectively alkyl and form together with the atoms to which they are attached a 3 to 8 membered ring system which may be interrupted by —O—, —S—, —NR₈—, or —CO—;
R₇ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl; or R₇ and R₅ are collectively alkyl and form together with the atoms to which they are attached a 5 to 10 membered ring system, or
R₇ and R₃ are collectively alkyl and form together with the atoms to which they are attached a 5 to 10 membered ring system;

Preferably, the variables R₁ through R₇ in the ligands of formula (a), (b) and (c) are:

R₁ is hydrogen, alkyl, or aryl(lower)alkyl or R₁ and R₂ are collectively alkyl and form together with the atoms to which they are attached a 4 to 8 membered ring system, or R₁ and R₄ are collectively alkyl and form together with the atoms to which they are attached a 4 to 8 membered ring system, or R₁ and R₆ are collectively alkyl and form together with the atoms to which they are attached a 4 to 8 membered ring system;
R₂ is hydrogen, alkyl, aryl(lower)alkyl or aryl, or R₂ and R₃ are collectively alkyl and form together with the atoms to which they are attached a 3 to 7 membered ring system, or R₂ and
R₄ are collectively alkyl and form together with the atoms to which they are attached a 5 to 10 membered ring system, or R₂ and R₆ are collectively alkyl and form together with the atoms to which they are attached a 5 to 10 membered ring system;
R₃ is hydrogen, alkyl, aryl(lower)alkyl or aryl, or R₃ and R₁ are collectively alkyl and form together with the atoms to which they are attached a 5 to 10 membered ring system, or R₃ and R₄ are collectively alkyl and form together with the atoms to which they are attached a 5 to 10 membered ring system, or R₃ and R₆ are collectively alkyl and form together with the atoms to which they are attached a 5 to 10 membered ring system, or R₃ and R₂ combine together to one oxygen atom of a carbonyl-group;
R₄ is hydrogen, alkyl, aryl(lower)alkyl, or aryl, or R₄ and R₅ are collectively alkyl and form together with the atoms to which they are attached a 3 to 7 membered ring system, or R₄ and
R₆ are collectively alkyl and form together with the atoms to which they are attached a 4 to 8 membered ring system, or R₄ and R₅ combine together to one oxygen atom of a carbonyl-group;
R₅ is hydrogen, alkyl, aryl(lower)alkyl, or aryl, or R₅ and R₆ are collectively alkyl and form together with the atoms to which they are attached a 4 to 8 membered ring system, or R₅ and R₃ are collectively alkyl and form together with the atoms to which they are attached a 4 to 8 membered ring system, or R₅ and R₆ are collectively alkyl and form together with the atoms to which they are attached a 4 to 8 membered ring system;
R₆ is hydrogen, alkyl or aryl(lower)alkyl, or R₆ and R₇ are collectively alkyl and form together with the atoms to which they are attached a 3 to 8 membered ring system which may be interrupted by —O—, —S—, —NR₈—, or —CO—; and
R₇ is hydrogen, alkyl, aryl(lower)alkyl, alkyl-carbonyl, or alkyloxy-carbonyl, or R₇ and R₅ are collectively alkyl and form together with the atoms to which they are attached a 5 to 10 membered ring system, or R₇ and R₃ are collectively alkyl and form together with the atoms to which they are attached a 5 to 10 membered ring system.

Preferably in a ligand of formula (a), (b) or (c) the substituents R₁ to R₆ are independently from each other hydrogen, alkyl, or aryl, and R₇ is hydrogen, alkyl, aryl(lower)alkyl, alkyl-carbonyl, or alkyloxy-carbonyl.

Also preferably in a ligand of formula (a), (b) or (c) the substituents R₁ to R₆ are independently from each other hydrogen, lower alkyl, or aryl, and R₇ is hydrogen, lower alkyl, or aryl(lower)alkyl.

In a preferred aspect, in a ligand of formula (a), (b) or (c) R₁ stands for hydrogen and R₂ to R₇ are independently selected from hydrogen and lower alkyl.

In another preferred aspect ligand X₁ is selected from the group of formulae (d) and (e).

Preferably any one (1) of the indices selected from q, r, s and t stands for 1 and the others are 0. In another preferred aspect, index s=1, and q, r, and t are all 0; in another preferred aspect index s=1 and q, r, and t are independently from each other 0 or 1; in another preferred aspect index s=1 and at least one of q, r, and t is 0; in another preferred aspect index s=1 and at least two of q, r, and t are 0; in another preferred aspect index s=1 and q, r, and t are all 0.

In a preferred aspect X₁ stands for 1-piperazinyl, 4-alkyl-1-piperazinyl, 1-homopiperazinyl or 4 alkyl-1-homopiperazinyl, wherein the piperazine and the homopiperazine ring may contain one or more lower alkyl substituents, and wherein alkyl preferably stands for lower alkyl and is methyl, ethyl or propyl, more preferably ethyl or methyl.

In a preferred aspect X₁ stands for N-piperidinyl which may be substituted in the 2-, 3- or 4-position by hydroxyl, amino, alkylamino, or dialkylamino.

In a preferred aspect X₁ is —NR₈-alkylene-N(alkyl)₂, —NR₈-alkylene-NH-alkyl, or —NR₈-alkylene-NH₂, wherein R₈ is hydrogen or lower alkyl, wherein alkyl is preferably lower alkyl and wherein alkylene is linear, branched, or cyclic and bonded in any position and is preferably lower alkylene with up to 7 carbon atoms.

In a preferred aspect X₁ is —O-alkylene-N(alkyl)₂, —O-alkylene-NH-alkyl, or —O-alkylene-NH₂, wherein alkyl is preferably lower alkyl and wherein alkylene is linear, branched, or cyclic and bonded in any position and is preferably lower alkylene with up to 7 carbon atoms.

In a preferred aspect X₁ is —NR₈-alkylene-OH or —NR₈-alkylene-O-alkyl, wherein R₈ is hydrogen or lower alkyl, wherein alkyl is preferably lower alkyl and wherein alkylene is linear, branched, or cyclic and bonded in any position and is preferably lower alkylene with up to 7 carbon atoms.

In another preferred aspect X₁ is —NR₈-alkylene-NH₂ and R₈ is hydrogen, wherein said alkylene is linear, branched, or cyclic and bonded in any position and is preferably lower alkylene with up to 7 carbon atoms.

In another preferred aspect X₁ is —NR₈—CH₂—CHR₁₁—NH₂, wherein R₈ is hydrogen or lower alkyl, preferably hydrogen, and wherein R₁₁ is lower alkyl, preferably ethyl or methyl, and wherein the carbon atom to which R₁₁ is attached may be racemic or chiral, preferably chiral, preferably in R-configuration, and preferably in S-configuration.

In another preferred aspect X₁ is —O—CH₂—CHR₁₁—NH₂, wherein R₈ is hydrogen or lower alkyl, preferably hydrogen, and wherein R₁₁ is lower alkyl, preferably ethyl or methyl, and wherein the carbon atom to which R₁₁ is attached may be racemic or chiral, preferably chiral, preferably in R-configuration, and preferably in S-configuration.

In another preferred aspect X₁ is —NR₈—CH₂—CHR₁₁—OH, wherein R₈ is hydrogen or lower alkyl, preferably hydrogen, and wherein R₁₁ is lower alkyl, preferably ethyl or methyl, and wherein the carbon atom to which R₁₁ is attached may be racemic or chiral, preferably chiral, preferably in R-configuration, and preferably in S-configuration.

R₂₃ is preferably selected from hydrogen, halogen, lower alkyl, and hydroxyl, even more preferably hydrogen, halogen, and lower alkyl, in particular hydrogen and halogen, most preferably hydrogen.

Preferably, R₂₀ and R₂₁ are independently selected from the group consisting of hydrogen, cyano, amino, N-alkylamino, N,N-dialkylamino, —NH-alkylene-aryl, —NH-aryl, halo and hydroxyl, more preferably from hydrogen, cyano, amino, N-alkylamino, and halogen, also preferably from hydrogen, amino, N-alkylamino, N,N-dialkylamino, —NH-alkylene-aryl, and —NH-aryl, more preferably from hydrogen, cyano, amino, and halogen.

### Bibliographic Convention

As used herein halogen or halo or vice versa and stands for fluorine, chlorine, bromine, or iodine, more preferably fluorine, or chlorine, highly preferably fluorine.

As used herein alkyl has up to 18 carbon atoms, is linear, branched, cyclic or a combination thereof and is preferably lower alkyl, more specifically methyl, ethyl, n-propyl, isopropyl, cyclopropyl, methylcyclopropyl, cyclopropylmethyl, cyclobutyl, iso-butyl, sec-butyl, tert-butyl, pentyl, iso-pentyl, neo-pentyl, hexyl, heptyl, octyl, nonyl, decyl, undecyl, dodecyl, terdecyl, quattordecyl, quindecyl and the like. Preferably lower alkyl stands for methyl, ethyl or propyl, in particular methyl.

As used herein alkylene has up to 18 carbon atoms, is linear, branched, or cyclic and bonded in any position and is preferably lower alkylene and is, for example, straight-chained or branched C₁-C₅alkylene, such as especially methylene, 1,2-ethylene, 1,3- or 1,2-propylene, 2,2-dimethylethylene, 1,1-dimethylethylene, 1,4-, 1,3- or 2,3-butylene, 1,5-, 1,4-pentylene, 1,1-cyclopropylethylene, 1,1-cyclopropylpropylene or the like.

As used herein alkoxy or Oalk has up to 18 carbon atoms, is linear, branched, is preferably lower alkoxy and is, for example, C₁-C₇alkoxy, preferably C₁-C₅alkoxy, such as methoxy, ethoxy, propyloxy, isopropyloxy or butyloxy, but may also be isobutyloxy, sec-butyloxy, tert-butyloxy or a pentyloxy, hexyloxy or heptyloxy group.

As used herein the term lower denotes a radical or a compound having from 1 up to 7 carbon atoms, preferably from 1 to 5 carbon atoms, in particular from 1 to 3 carbon atoms and especially from 1-2 carbon atoms.

As used herein, aryl stands for an aromatic moiety having from 6 to 14 carbon atoms, and is for example, phenyl or naphthyl that is unsubstituted or substituted by lower alkyl, lower alkoxy, hydroxy, lower alkoxycarbonyl, carboxy, carbamoyl, sulfamoyl, lower alkanoyl, halogen and/or by trifluoromethyl. Aryl as used herein stands also for an unsubstituted or substituted heteroaromatic radical optionally partially hydrogenated, 5- or 6-membered monocyclic heteroaryl or bicyclic heteroaryl composed of 5- or 6-membered rings, such as corresponding furyl, lower alkylfuryl, for example 4-methylfur-2-yl, thienyl, imidazolyl, for example imidazol-4-yl, oxazolyl, carboxy-lower alkyl(oxo)oxazolyl, for example 2,5-dihydro-3-oxo-1,2-oxazolyl, thiazolyl, dihydrothiazolyl, for example 4,5-dihydrothiazolyl, carboxy-lower alkylthiazolyl, for example 4-carboxymethylthiazolyl, lower alkoxycarbonyl-lower alkylthiazolyl, for example 4-methoxycarbonylmethylthiazolyl or 4-ethoxycarbonyl-methylthiazolyl, tetrazolyl, pyridyl, pyrazinyl, indolyl, for example indol-3-yl, quinolinyl, for example quinolin-4-yl, benzazepinyl or carboxy-lower alkyl-2,3,4,5-tetrahydro-1H-1-benzazepino, for example 1-carboxymethyl-2,3,4,5-tetrahydro-1H-1-benzazepino. Preferably, aryl is phenyl or naphthyl that is unsubstituted or substituted by lower alkyl, lower alkoxy, hydroxy, carboxy, carbamoyl, sulfamoyl, lower alkanoyl, halogen and/or by trifluoromethyl. Even more preferably aryl is phenyl or naphthyl that is unsubstituted or substituted by lower alkyl, halogen, lower alkoxy or hydroxy.

### Salts:

The compounds of the present invention, e.g. a compound of formula I, may exist in free form or in salt form, e.g. salts with organic or inorganic acids, especially pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, glycolic acid, lactic acid, 2-hydroxybutyric acid, gluconic acid, glucosemonocarboxylic acid, fumaric acid, succinic acid, adipic acid, pimelic acid, malic acid, tartaric acid, citric acid, or other organic protonic acids, such as ascorbic acid, or a mixture thereof.

### Process of Manufacture

### Method A:

The present invention also provides a process for the production of a compound of general formula I being obtainable by the reaction steps of:

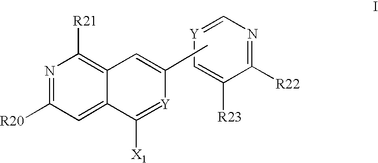

A halogenated [2,6]naphthyridine of formula Iₐ wherein Y═N and the coreactant X₁H are reacted with each other, typically for several hours, typically at elevated temperature and preferably in a solvent, e.g. in 1-Methyl-pyrrolidin-2-one as solvent;

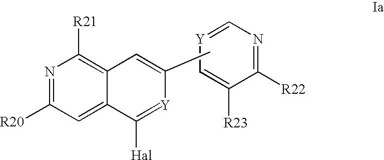

wherein the compound of formula Ia may be obtainable from reacting a mixture of a hydroxyl-compound in accordance to formula Ib and a halogenating agent, e.g. POCl₃, are reacted, e.g. heated to 80° C. for 24 h;

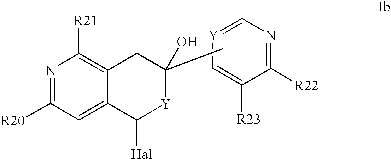

wherein the compound of formula Ib may be obtainable from reacting a lactone of formula Ic with a reagent YH₃, which stands for ammonia (NH₃);

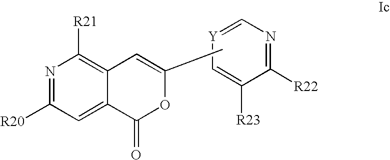

wherein the compound of formula Ic may be obtainable from reacting a compound of formula Id with a dehydrating agent such as for example treating in a microwave oven,

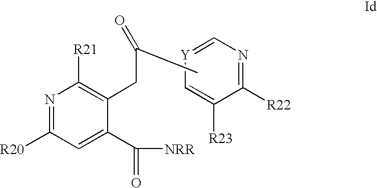

wherein the variables have the meaning indicated above.

### Method B:

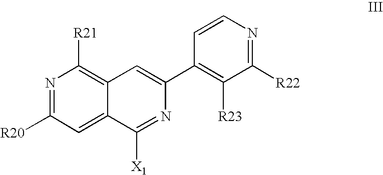

The present invention also provides a one step process for the production of a compound of general formula III, wherein the variables have the definitions rendered above, being obtainable by:

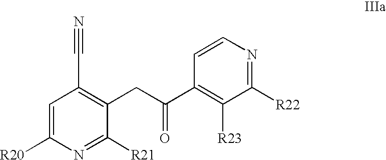

Reacting a pyridylnitrile compound of formula IIIa and typically in a protic solvent such as, methanol, ethanol, acidic acid and the like and preferably in the presence of a Lewis acid catalyst such as SiO₂, Al₂O₃ or the like with a ligand of formula (a), (b), (c), (d) or (e), wherein the variables have the definitions rendered above, and wherein the free bond in such a ligand is attached to a hydrogen atom.

### EXPERIMENTAL PART

Insofar as the production of the starting materials is not particularly described, the compounds are known or may be prepared analogously to methods known in the art or as described hereafter.

The following examples are illustrative of the invention without any limitation.

### ABBREVIATIONS

BINAP racemic 2,2′-bis(diphenylphosphino)-1,1′-binaphthyl
bs broad singlet
d doublet
dd doublet of doublets
DIPEA N-ethyldiisopropylamine
DME 1,4-dimethoxyethane
DMF N,N-dimethylformamide
DMSO dimethylsulfoxide
EtOAc ethyl acetate
FCC flash column chromatography
HPLC high pressure liquid chromatography
MeOH methanol
MS mass spectroscopy
MW microwave
m multiplet
NMR nuclear magnetic resonance
rt room temperature
s singulet
t triplet
TFA trifluoroacetic acid
THF tetrahydrofuran

All compounds are named using AutoNom.

### Example 1

### 2-Methyl-N*1*-(3-pyridin-4-yl-[2,6]naphthyridin-1-yl)-propane-1,2-diamine

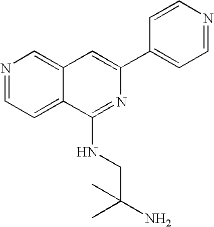

### Method A

A solution of 1-chloro-3-pyridin-4-yl-[2,6]naphthyridine (150 mg, 0.559 mmol) in 1-methyl-1,2-diaminopropane (2 mL) is stirred for 5 h at 90° C. (alternatively, 1-Methyl-pyrrolidin-2-one is used as solvent). The reaction mixture is cooled to rt and concentrated under reduced pressure. The residue is purified by FCC (SiO₂, gradient elution, CH₂Cl₂/CH₂Cl₂:NH₃ (2 Min MeOH) (9:1) 100:0→0:100, 30 min) to yield the title compound (145 mg, 0.470 mmol, 84%) as a pale yellow foam. ¹H-NMR (400 MHz, DMSO-d₆, 298 K): 1.11 (s, 6H), 1.78 (s, NH), 3.61 (d, 2H), 7.62 (t, NH), 7.83 (s, 1H), 8.10 (d, 2H), 8.26 (d, 1H), 8.61 (d, 1H), 8.67 (d, 2H), 9.20 (s, 1H). MS: 294.2 [M+1]⁺. Alternatively, the crude product is purified by preparative reverse-phase HPLC (Waters) to give the title compound as the TFA salt.

### 1-Chloro-3-pyridin-4-yl-[2,6]naphthyridine

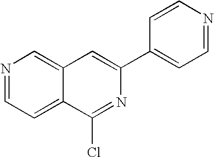

A suspension of 3-hydroxy-3-pyridin-4-yl-3,4-dihydro-2H-[2,6]naphthyridin-1-one (3.00 g, 11.8 mmol) in POCl₃ (50 mL) is heated to 80° C. for 24 h. The reaction mixture is concentrated under reduced pressure to remove excess of POCl₃. The residual oil is treated with ice-cold H₂O and the suspension thus obtained is basified to pH14 with 10 N NaOH while keeping the temperature below rt. The mixture is filtered and the aqueous filtrate is extracted with CH₂Cl₂ (2×). The combined organic layers are dried over MgSO₄, filtered, and evaporated to dryness to yield a first crop of the crude title compound. The gluey precipitate obtained from the filtration is extracted with CH₂Cl₂ (stirring for 10 min at rt, 2×) to yield a second crop of the crude title compound. The combined crude products are purified by FCC (SiO₂, gradient elution, CH₂Cl₂/MeOH 100:0→94:6, 35 min) to yield the title compound (932 mg, 3.78 mmol, 32%) as a white solid. ¹H-NMR (400 MHz, DMSO-d₆, 298 K): 8.10-8.12 (m, 3H), 8.75-8.77 (m, 2H), 8.87-8.91 (m, 2H), 9.56 (s, 1H). MS: 242.2 [M+1]⁺.

### 1-Bromo-3-pyridin-4-yl-[2,6]naphthyridine

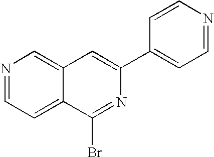

By using POBr₃ (150° C., 6 h) instead of POCl₃ the corresponding bromo derivative can be obtained and used in the same way as the chloro derivative.

### 3-Hydroxy-3-pyridin-4-yl-3,4-dihydro-2H-[2,6]naphthyridin-1-one

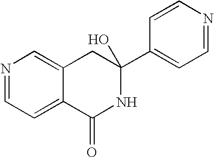

A suspension of 3-pyridin-4-yl-pyrano[4,3-c]pyridin-1-one (6.70 g, 28.4 mmol) in NH₃ (7 M in MeOH) is stirred for 2 h at rt. The reaction mixture is evaporated to dryness to yield the title compound (7.12 g, 28.0 mmol, 99%) as a white solid. ¹H-NMR (400 MHz, DMSO-d₆, 298 K): 3.13-3.15 (m, 1H), 3.34-3.38 (m, 1H), 6.72 (s, OH), 7.58 (d, 2H), 7.79 (d, 1H), 8.59-8.61 (m, 3H), 8.65 (d, 1H), 9.11 (s, NH). MS: 242.3 [M+1]⁺.

### 3-Pyridin-4-yl-pyrano[4,3-c]pyridin-1-one

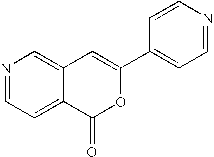

A solution of N-tert-butyl-3-(2-oxo-2-pyridin-4-yl-ethyl)-isonicotinamide (6.50 g, 20.8 mmol) in DMF (12 mL) is heated to 220° C. for 5 min in a microwave oven. The product, 3-pyridin-4-yl-pyrano[4,3-c]pyridin-1-one, precipitates from the reaction mixture and is isolated by filtration. The filtrate is heated two more times to 220° C. for 5 min in a microwave oven to give two other crops of the product. The precipitates are combined to yield the title compound (4.05 g, 17.2 mmol, 83%) as a white solid. ¹H-NMR (400 MHz, DMSO-d₆, 298 K): 7.84-7.87 (m, 3H), 8.04 (d, 1H), 8.74 (d, 2H), 8.83 (d, 1H), 9.09 (s, 1H). MS: 225.1 [M+1]⁺.

### N-tert-Butyl-3-(2-oxo-2-pyridin-4-yl-ethyl)isonicotinamide

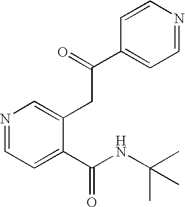

To a solution of N-tert-butyl-3-methyl-isonicotinamide (10.4 g, 51.4 mmol) in THF (220 mL) is added BuLi (69.0 mL, 110 mmol, 1.6 M in hexanes) at −45° C. under inert atmosphere. The reaction mixture is stirred for 60 min at −45° C. (a bright red suspension is obtained), and then isonicotinic acid methyl ester (6.54 mL, 54.8 mmol) is added in one portion. The cooling bath is removed and stirring is continued for 2 h at rt. The reaction mixture is diluted with EtOAc and washed with saturated aqueous NH₄Cl solution. The organic layer is separated and the aqueous layer is extracted with EtOAc (3×). The combined organic layers are dried over MgSO₄, filtered, and evaporated to dryness. The residue is purified by FCC (SiO₂, gradient elution, EtOAc/MeOH 100:0→90:10, 25 min) to yield the title compound (11.6 g, 37.1 mmol, 72%) as a pale yellow solid. ¹H-NMR (400 MHz, DMSO-d₆, 298 K): 1.21 (s, 9H), 4.63 (d, 2H), 7.38 (d, 1H), 7.86 (d, 2H), 8.11 (s, NH), 8.51 (s, 1H), 8.56 (d, 1H), 8.83 (d, 2H). MS: 298.2 [M+1]⁺.

### N-tert-Butyl-3-methyl-isonicotinamide

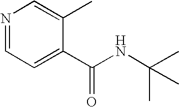

To a suspension of 3-methyl-isonicotinic acid (10.0 g, 72.2 mmol) in CH₂Cl₂ (300 mL) is added oxalylchloride (9.30 mL, 108 mmol) and DMF (1 drop) at rt. The reaction mixture is stirred for 30 min at rt (a clear brown solution is obtained) and then concentrated under reduced pressure. The solid residue is suspended in CH₂Cl₂ (150 mL) and treated with Et₃N (12.1 mL, 86.6 mmol) and tert-butylamine (8.08 mL, 75.8 mmol) at 0° C. The cooling bath is removed and stirring is continued for 12 h at rt. The reaction mixture is diluted with CH₂Cl₂ and washed with 1 N aqueous NaOH. The organic layer is dried over MgSO₄, filtered, and evaporated to dryness to yield the title compound (10.4 g, 51.4 mmol, 71%) as a brown solid, which is used without further purification for the next step. ¹H-NMR (400 MHz, DMSO-d₆, 298 K): 1.35 (s, 9H), 2.27 (s, 3H), 7.18 (d, 1H), 8.03 (s, NH), 8.40 (d, 1H), 8.44 (s, 1H).

### Method B

To a solution of 3-(2-oxo-2-pyridin-4-yl-ethyl)-isonicotinonitrile (200 mg, 0.887 mmol) in a mixture of EtOAc/acetic acid 6:4 (15.0 mL) is added 2-methyl-propane-1,2-diamine (1.0 mL). Silica gel 60 (3.0 g) is added and the reaction mixture is stirred for 2 h at rt. The reaction mixture is filtered and the residue washed with EtOAc. The filtrate is concentrated by rotary evaporation. The residue is partitioned between EtOAc and an aqueous 1 M NaOH solution. The organic phase is dried over Na₂SO₄, filtered and concentrated at reduced pressure. Purification by FCC (silica gel, CH₂Cl₂/EtOH, 33% aqueous NH₃ 80:18:2) affords the title compound as yellow crystals (122 mg, 0.416 mmol, 47%). ¹H NMR (400 MHz, DMSO-d₆, 298 K): δ=9.18 (s, 1H), 8.66 (d, J=6.1 Hz, 2H), 8.59 (d, J=5.8 Hz, 1H), 8.24 (d, J=5.8 Hz, 1H), 8.08 (d, J=6.1 Hz, 2H), 7.81 (s, 1H), 7.60 (bs, 1H), 3.61 (d, J=5.5 Hz, 2H), 1.66 (bs, 2H), 1.11 (s, 6H). MS (ES⁺): 294 (M(C₁₇H₁₉N₅)+H)⁺.

### 3-(2-Oxo-2-pyridin-4-yl-ethyl)isonicotinonitrile

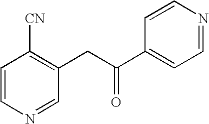

In a three-necked round-bottomed flask equipped with reflux cooler, 60% NaH in mineral oil (4.06 g, 102 mmol) is added to DME (80 mL). The suspension is heated to 95° C. and a solution of 3-methyl-isonicotinonitrile (3.00 g, 25.4 mmol) and isonicotinic acid methyl ester (3.48 g, 25.4 mmol) in DME (20 mL) is added. The resulting reaction mixture is stirred overnight at 95° C. After cooling down to room temperature, the reaction mixture is partitioned between EtOAc and water. The aqueous phase is extracted with EtOAc and the combined organic layers are dried over Na₂SO₄, filtered and concentrated in vacuo to afford an orange solid (5.67 g, 25.4 mmol, 100%). ¹H NMR (400 MHz, DMSO-d₆, 298 K): δ=9.19 (s, 1H), 8.72 (d, J=6.2 Hz, 2H), 8.65 (d, J=5.1 Hz, 1H), 7.95 (d, J=5.1 Hz, 1H), 7.84 (d, J=6.2 Hz, 2H), 4.84 (s, 2H).

### 3-Methyl-isonicotinonitrile

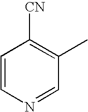

To 3-methyl-pyridine 1-oxide (15.9 g, 150 mmol) is added at 0° C. during 30 min. dimethylsulfate (15.6 mL). The resulting reaction mixture is stirred overnight at 40° C. A solution of KCN (10.75 g, 165 mmol) in a mixture of EtOH/water 1:1 (120 mL) is added and the reaction mixture is stirred overnight at 40° C. The reaction mixture is concentrated in vacuo and the residue is partitioned between EtOAc and water. The aqueous phase is extracted with EtOAc and the combined organic layers are dried over Na₂SO₄, filtered and concentrated at reduced pressure. Purification by FCC (silica gel, cyclohexane/EtOAc 85:15) affords the title compound as orange crystals (6.0 g, 50.8 mmol, 34%). ¹H NMR (400 MHz, DMSO-d₆, 298 K): δ=8.76 (s, 1H), 8.64 (d, J=4.9 Hz, 1H), 7.80 (d, J=4.9 Hz, 1H).

By following the procedures of Example 1, but by using the appropriate starting materials, the compounds of formula A wherein R is as indicated in Table 1 below may be obtained.

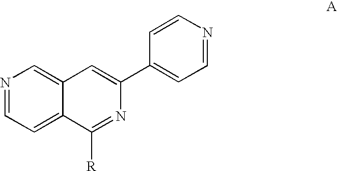

TABLE 1

| | name | R | MS |
|---|---|---|---|
| 2. | N*1*-(3-Pyridin-4-yl-[2,6]naphthyridin-1- yl)-ethane-1,2-diamine | 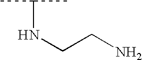 | MH⁺ 266.2 |
| 3. | N,N-Dimethyl-N′-(3-pyridin-4-yl- [2,6]naphthyridin-1-yl)-ethane-1,2- diamine | 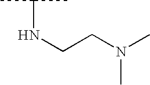 | MH⁺ 294.2 |
| 4. | N-Isopropyl-N′-(3-pyridin-4-yl- [2,6]naphthyridin-1-yl)-ethane-1,2- diamine | 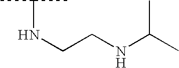 | MH⁺ 308.3 |
| 5. | N-Methyl-N′-(3-pyridin-4-yl- [2,6]naphthyridin-1-yl)-ethane-1,2- diamine | 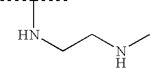 | MH⁺ 280.2 |
| 6. | N,N,N′-Trimethyl-N′-(3-pyridin-4-yl- [2,6]naphthyridin-1-yl)-ethane-1,2- diamine | 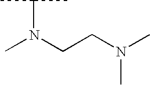 | MH⁺ 308.2 |
| 7. | 3-Phenyl-N*1*-(3-pyridin-4-yl- [2,6]naphthyridin-1-yl)-propane-1,2- diamine | 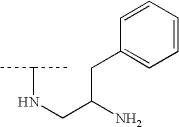 | MH⁺ 356.2 |
| 8. | (1-Ethyl-pyrrolidin-2-ylmethyl)-(3- pyridin-4-yl-[2,6]naphthyridin-1-yl)- amine | 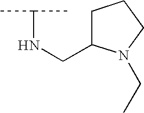 | MH⁺ 334.3 |
| 9. | (2-Piperidin-1-yl-ethyl)-(3-pyridin-4-yl- [2,6]naphthyridin-1-yl)-amine | 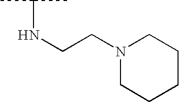 | MH⁺ 334.3 |
| 10. | (1-Methyl-piperidin-4-yl)-(3-pyridin-4-yl- [2,6]naphthyridin-1-yl)-amine | 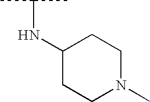 | MH⁺ 320.3 |
| 11. | N,N-Dimethyl-N′-(3-pyridin-4-yl- [2,6]naphthyridin-1-yl)-propane-1,3- diamine | 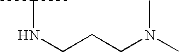 | MH⁺ 308.3 |
| 12. | Dimethyl-[1-(3-pyridin-4-yl- [2,6]naphthyridin-1-yl)-piperidin-4-yl]- amine | 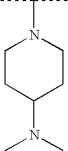 | MH⁺ 334.3 |
| 13. | 1-[1,4]Diazepan-1-yl-3-pyridin-4-yl- [2,6]naphthyridine | 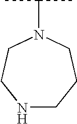 | MH⁺ 306.3 |
| 14. | 2-Methyl-N*1*-(3-pyridin-4-yl- [2,6]naphthyridin-1-yl)-propane-1,3- diamine | 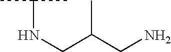 | MH⁺ 294.2 |
| 15. | 2,2-Dimethyl-N*1*-(3-pyridin-4-yl-[2,6] naphthyridin-1-yl)-propane-1,3- diamine | 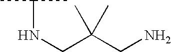 | MH⁺ 308.2 |
| 16. | Rac-(1R,3R)-N-(3-Pyridin-4-yl-[2,6]naph thyridin-1-yl)-cyclohexane-1,3-diamine | 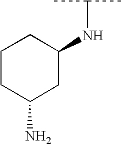 | MH⁺ 320.4 |
| 17. | Rac-(1R,3S)-N-(3-Pyridin-4-yl-[2,6]naph thyridin-1-yl)-cyclohexane-1,3-diamine | 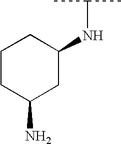 | MH⁺ 320.4 |
| 18. | 4-(3-Pyridin-4-yl-[2,6]naphthyridin- 1-yl)-1,4-diaza-spiro[5.5]undecane | 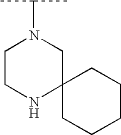 | MH⁺ 360.5 |
| 19. | 1-(3,3-Dimethyl-piperazin-1-yl)-3-py- ridin-4-yl-[2,6]naphthyridine | 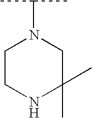 | MH⁺ 320.2 |
| 20. | 1-(3-Methyl-piperazin-1-yl)-3-pyridin-4- yl-[2,6]naphthyridine | 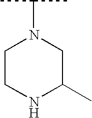 | MH⁺ 306.2 |
| 21. | 1-((R)-3-Methyl-piperazin-1-yl)-3- pyridin-4-yl-[2,6]naphthyridine | 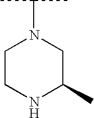 | MH⁺ 306.2 |
| 22. | 1-((S)-3-Methyl-piperazin-1-yl)-3- pyridin-4-yl-[2,6]naphthyridine | 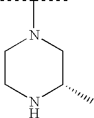 | MH⁺ 306.2 |
| 23. | Rac-1-((2S,5R)-2,5-Dimethyl-piperazin- 1-yl)-3-pyridin-4-yl-[2,6]naphthyridine | 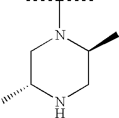 | MH⁺ 320.2 |
| 24. | Rac-1-((2S,5S)-2,5-Dimethyl-piperazin- 1-yl)-3-pyridin-4-yl-[2,6]naphthyridine | 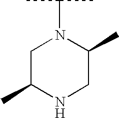 | MH⁺ 320.2 |
| 25. | 1-((2S,5R)-2,5-Dimethyl-piperazin-1-yl)- 3-pyridin-4-yl-[2,6]naphthyridine | 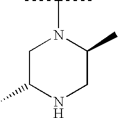 | MH⁺ 320.2 |
| 26. | 1-((2R,5S)-2,5-Dimethyl-piperazin-1-yl)- 3-pyridin-4-yl-[2,6]naphthyridine | 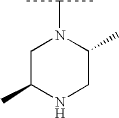 | MH⁺ 320.2 |
| 27. | 1-(3,5-Dimethyl-piperazin-1-yl)-3- pyridin-4-yl-[2,6]naphthyridine | 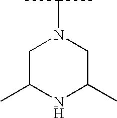 | MH⁺ 320.2 |
| 28. | 1-(3-Phenyl-piperazin-1-yl)-3-pyridin-4- yl-[2,6]naphthyridine | 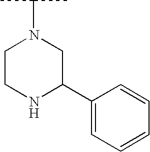 | MH⁺ 368.2 |
| 29. | 4-(3-Pyridin-4-yl-[2,6]naphthyridin-1-yl)- piperazin-2-one | 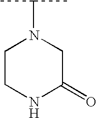 | MH⁺ 306.1 |
| 30. | 1-(4-Methyl-piperazin-1-yl)-3-pyridin-4- yl-[2,6]naphthyridine | 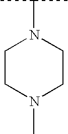 | NH⁺ 306.2 |
| 31. | 1-[4-(3-Pyridin-4-yl-[2,6]naphthyridin-1- yl)-piperazin-1-yl]-ethanone | 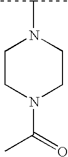 | MH⁺ 334.2 |
| 32. | N-(3-Pyridin-4-yl-[2,6]naphthyridin-1-yl)- cyclohexane-1,4-diamine | 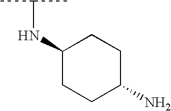 | MH⁺ 320.2 |
| 33. | [3-(4-Methyl-piperazin-1-yl)-propyl]-(3- pyridin-4-yl-[2,6]naphthyridin-1-yl)- amine | 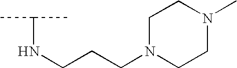 | MH⁺ 363.3 |
| 34. | 3-(3-Pyridin-4-yl-[2,6]naphthyridin-1- ylamino)-propan-1-ol | 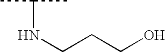 | MH⁺ 281.3 |
| 35. | 2-(3-Pyridin-4-yl-[2,6]naphthyridin-1- ylamino)-ethanol | 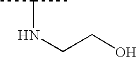 | MH⁺ 267.5 |
| 36. | 1-(3-Pyridin-4-yl-[2,6]naphthyridin-1-yl)- piperidin-4-ol | 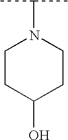 | MH⁺ 307.4 |
| 37. | 2-(3-Pyridin-4-yl-[2,6]naphthyridin-1- ylamino)-ethanol | 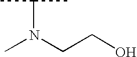 | MH⁺ 281.3 |
| 38. | 2-(3H-Imidazol-4-yl)-ethyl]-(3-pyridin-4- yl-[2,6]naphthyridin-1-yl)-amine | 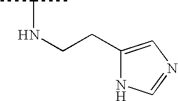 | MH⁺ 317.4 |
| 39. | (3-Pyridin-4-yl-[2,6]naphthyridin-1-yl)- (3-pyrrol-1-yl-propyl)-amine | 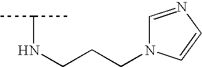 | MH⁺ 331.4 |
| 40. | 1-(4,7-diaza-spiro[2.5]oct-7-yl)-3-pyridin- 4-yl-[2,6]naphthyridine | 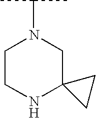 | MH⁺ 318.3 |

By following the procedures of Example 1, but by using the appropriate starting materials, the compounds of formula B wherein R is as indicated in Table 2 below may be obtained.

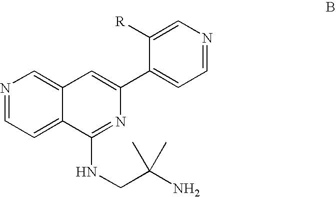

TABLE 2

| | name | R | MS |
|---|---|---|---|
| 41. | N*1*-[3-(3-Fluoro-pyridin-4-yl)-[2,6]naph- | F | MH⁺ 312.2 |
| | thyridin-1-yl]-2-methyl-propane-1,2-diamine | | |
| 42. | N*1*-[3-(3-Chloro-pyridin-4-yl)-[2, | Cl | MH⁺ 328.1 |
| | 6]naphthyridin-1-yl]-2-methyl-propane | | |
| | -1,2-diamine | | |
| 43. | 2-Methyl-N*1*-[3-(3-methyl-pyridin-4- | Me | MH⁺ 308.2 |
| | yl)-[2,6]naphthyridin-1-yl]-propane-1,2- | | |
| | diamine | | |

### Example 44

### 1-(3-Pyridin-4-yl-[2,6]naphthyridin-1-yl)-piperidin-4-ylamine

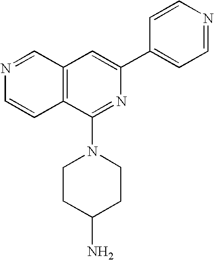

A solution of [1-(3-pyridin-4-yl-[2,6]naphthyridin-1-yl)-piperidin-4-yl]-carbamic acid tert-butyl ester in TFA (0.5 mL) and CH₂Cl₂ (0.5 mL) is stirred for 1 h at rt. The reaction mixture is concentrated under reduced pressure. The residue is purified by preparative reverse phase HPLC (Waters) to yield the title compound (19 mg, 0.036 mmol, 18% over two steps, 2TFA salt) as a yellow solid. ¹H-NMR (400 MHz, DMSO-d₆, 298 K): 1.78-1.91 (m, 2H), 2.08 (d, 2H), 3.21 (t, 2H), 3.34-3.41 (m, 1H), 4.12 (d, 2H), 7.92 (d, 1H), 8.02 (s, NH), 8.40 (d, 2H), 8.47 (s, 1H), 8.72 (d, 1H), 8.86 (d, 2H), 9.43 (s, 1H). MS: 306.3 [M+1]⁺.

### [1-(3-Pyridin-4-yl-[2,6]naphthyridin-1-yl)-piperidin-4-yl]-carbamic acid tert-butyl ester

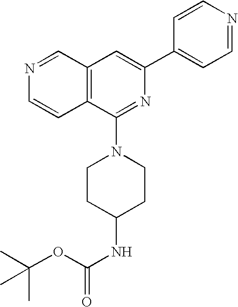

To a solution of 1-chloro-3-pyridin-4-yl-[2,6]naphthyridine (50.0 mg, 0.197 mmol) in DMF (0.5 mL) is added piperidin-4-yl-carbamic acid tert-butyl ester (83.0 mg, 0.394 mmol) and K₂CO₃ (55.0 mg, 0.394 mmol) at rt. The reaction mixture is heated to 90° C. and stirred for 5 h. (Alternatively, the two reagents are heated at 90° C. for 3-5 h using pure 1-methyl-pyrrolidin-2-one as solvent and DIPEA as base). The reaction mixture is cooled to rt, diluted with EtOAc and washed with H₂O. The organic layer is separated and the aqueous layer is extracted with EtOAc (3×). The combined organic layers are dried over MgSO₄, filtered, and evaporated to dryness. The residue is purified by FCC (SiO₂, gradient elution, CH₂Cl₂/MeOH 100:0→90:10, 26 min) to yield the title compound as a pale yellow solid.

By following the procedures of Example 44, but by using the appropriate starting materials, the compounds of formula A wherein R is as indicated in Table 3 below may be obtained.

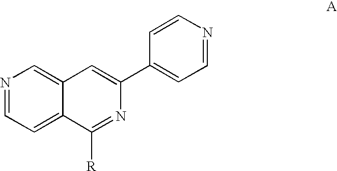

TABLE 3

| | name | R | MS |
|---|---|---|---|
| 45. | N*1*-(3-Pyridin-4-yl-[2,6]naphthyridin-1- yl)-propane-1,2-diamine | 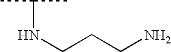 | MH⁺280.4 |
| 46. | N*1*-(3-Pyridin-4-yl-[2,6]naphthyridin-1- yl)-butane-1,2-diamine | 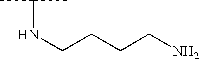 | MH⁺ 294.4 |
| 47. | N*1*-(3-Pyridin-4-yl-[2,6]naphthyridin-1- yl)-pentane-1,2-diamine | 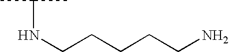 | MH⁺ 309.4 |
| 48. | N*1*-(3-Pyridin-4-yl-[2,6]naphthyridin-1- yl)-hexane-1,2-diamine | 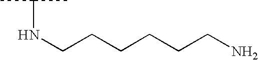 | MH⁺ 322.3 |
| 49. | 1-Piperazin-1-yl-3-pyridin-4-yl- [2,6]naphthyridine | 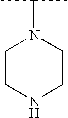 | MH⁺ 292.3 |
| 50. | (S)-1-Piperidin-2-ylmethyl-(3-pyridin-4- yl-[2,6]naphthyridin-1-yl)-amine | 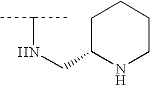 | MH⁺ 320.3 |
| 51. | (3-Pyridin-4-yl-[2,6]naphthyridin-1-yl)- pyrrolidin-2-ylmethyl-amine | 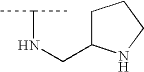 | MH⁺ 306.3 |
| 52. | 1-(3-Pyridin-4-yl-[2,6]naphthyridin- 1-yl)-piperidin-3-ylamine | 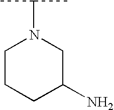 | MH⁺ 306.4 |
| 53. | N*1*-Methyl-N*1*-(3-pyridin-4-yl-[2,6] naphthyridin-1-yl)-ethane-1,2-di amine | 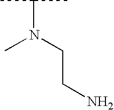 | MH⁺ 280.2 |
| 54. | N*1*-Ethyl-N*1*-(3-pyridin-4-yl-[2, 6]naphthyridin-1-yl)-ethane-1,2-di amine | 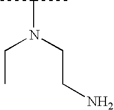 | MH⁺ 294.2 |
| 55. | N*1*-Isopropyl-N*1*-(3-pyridin-4-yl- [2,6]naphthyridin-1-yl)-ethane-1,2- diamine | 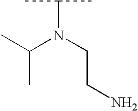 | MH⁺ 308.2 |
| 56. | N*1*-Methyl-N*1*-(3-pyridin-4-yl- 2[2,6]naphthyridin-1-yl)-propane-1,3- diamine | 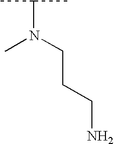 | MH⁺ 294.2 |
| 57. | N*1*-Ethyl-N*1*-(3-pyridin-4-yl- [2,6]naphthyridin-1-yl)-propane-1,3- diamine | 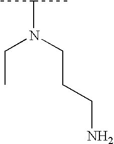 | MH⁺ 308.2 |
| 58. | N*1*-(3-Pyridin-4-yl-[2,6]naphthyridin-1- yl)-butane-1,3-diamine | 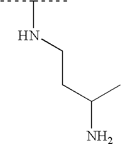 | MH⁺ 294.2 |
| 59. | 4-Phenyl-N*1*-(3-pyridin-4-yl- [2,6]naphthyridin-1-yl)-butane-1,3- diamine | 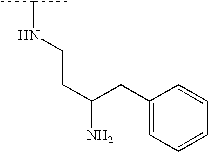 | MH⁺ 370.2 |
| 60. | (1-Amino-cyclopentylmethyl)-(3-pyri din-4-yl-[2,6]naphthyridin-1-yl)-amine | 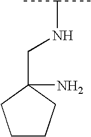 | MH⁺ 320.3 |
| 61. | (1-Amino-cyclohexylmethyl)-(3-pyridin- 4-yl-[2,6]naphthyridin-1-yl)-amine | 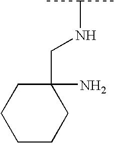 | MH⁺ 334.4 |
| 62. | 1-(3-Pyridin-4-yl-[2,6]naphthyridin- 1-yl)-piperidin-4-ylamine | 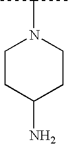 | MH⁺ 306.3 |
| 63. | C-[1-(3-Pyridin-4-yl-[2,6]naphthyri din-1-yl)-piperidin-3-yl]-methylamine | 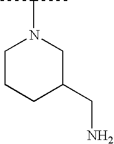 | MH⁺ 320.2 |
| 64. | 2-[1-(3-Pyridin-4-yl-[2,6]naphthyridin-1- yl)-piperidin-2-yl]-ethylamine | 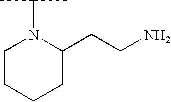 | MH⁺ 334.2 |
| 65. | 1-(3-Pyridin-4-yl-[2,6]naphthyridin- 1-yl)-pyrrolidin-3-ylamine | 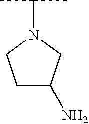 | MH⁺ 292.3 |
| 66. | Azetidin-3-yl-(3-pyridin-4-yl-[2,6] naphthyridin-1-yl)-amine | 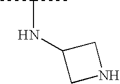 | MH⁺ 278.3 |
| 67. | Piperidin-3-yl-(3-pyridin-4-yl- [2,6]naphthyridin-1-yl)-amine | 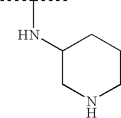 | MH⁺ 306.2 |
| 68. | 1-(2,5-Diaza-bicyclo[2.2.1]hept-2-yl)-3- pyridin-4-yl-[2,6]naphthyridine | 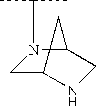 | MH⁺ 304.2 |
| 69. | Piperiidn-4-yl-(3-pyridin-4-yl-[2,6] naphthyridin-1-yl)-amine | 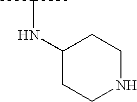 | MH⁺ 306.2 |
| 70. | 2-(3-Pyridin-4-yl-[2,6]naphthyridin- 1-ylsulfanyl)-ethylamine | 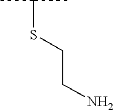 | MH⁺ 283.1 |

### Example 71

### Piperidin-4-ylmethyl-(3-pyridin-4-yl-[2,6]naphthyridin-1-yl)-amine

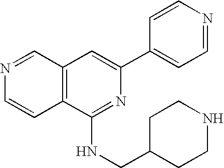

A solution of 4-[(3-pyridin-4-yl-[2,6]naphthyridin-1-ylamino)-methyl]-piperidine-1-carboxylic acid tert-butyl ester in TFA (0.5 mL) and CH₂Cl₂ (0.5 mL) is stirred for 2 h at rt. The reaction mixture is concentrated under reduced pressure. The residue is purified by preparative reverse phase HPLC (Waters) to yield the title compound (17 mg, 0.025 mmol, 13% over two steps, 2TFA salt) as a yellow foam. MS: 320.3 [M+1]⁺4-[(3-Pyridin-4-yl-[2,6]naphthyridin-1-ylamino)-methyl]piperidine-1-carboxylic acid tert-butyl ester.

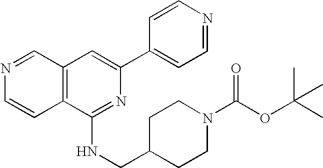

To a solution of 1-chloro-3-pyridin-4-yl-[2,6]naphthyridine (50.0 mg, 0.197 mmol) in 1,4-dioxane (5 mL) is added 4-aminomethyl-piperidine-1-carboxylic acid tert-butyl ester (128 mg, 0.590 mmol) and 40% aqueous NaOH (29.0 μL, 0.290 mmol) at rt. The reaction mixture is heated to 100° C. for 48 h, and then cooled to rt, diluted with EtOAc and filtered through a Florisil plug. The filtrate is evaporated to dryness to yield the title compound as a yellow oil, which is used without further purification for the next step.

### Example 72

### (S)—N*1*-(3-Pyridin-4-yl-[2,6]naphthyridin-1-yl)-propane-1,2-diamine and (S)—N*2*-(3-Pyridin-4-yl-[2,6]naphthyridin-1-yl)-propane-1,2-diamine

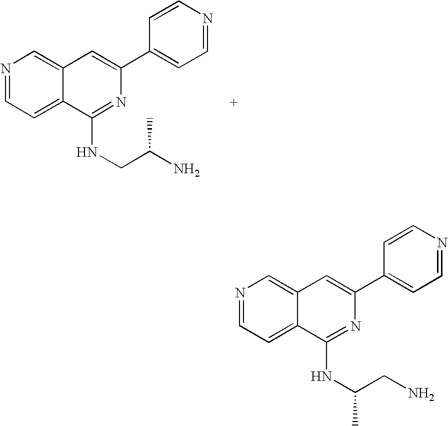

To a solution of 1-chloro-3-pyridin-4-yl-[2,6]naphthyridine (200 mg, 0.778 mmol) in 1,4-dioxane (4 mL) is added (S)-(+)-1,2-diaminopropane dihydrochloride (229 mg, 1.56 mmol) and 40% aqueous NaOH (390 μL, 3.90 mmol) at rt. The reaction mixture is heated to 100° C. for 36 h. The reaction mixture is cooled to rt, diluted with EtOAc and washed with 1 N NaOH. The organic layer is separated and the aqueous layer is extracted with EtOAc (2×). The combined organic layers are dried over MgSO₄, filtered, and evaporated to dryness. The residue is purified by preparative reverse phase HPLC (Waters) to yield the title compounds (S)—N*1*-(3-Pyridin-4-yl-[2,6]naphthyridin-1-yl)-propane-1,2-diamine (122 mg, 0.240 mmol, 31%, 2TFA salt) and (S)—N*2*-(3-Pyridin-4-yl-[2,6]naphthyridin-1-yl)-propane-1,2-diamine (22 mg, 0.043 mmol, 6%, 2TFA salt) as yellow solids. (S)—N*1*-(3-Pyridin-4-yl -[2,6]naphthyridin-1-yl)-propane-1,2-diamine (example 71a): ¹H-NMR (400 MHz, CD₃OD, 298 K): 1.48 (d, 3H), 3.75-4.05 (m, 3H), 8.18 (d, 1H), 8.24 (s, 1H), 8.76 (d, 1H), 8.83 (d, 2H), 8.91 (d, 2H), 9.37 (s, 1H). MS: 280.3 [M+1]⁺. (S)—N*2*-(3-Pyridin-4-yl-[2,6]naphthyridin-1-yl)-propane-1,2-diamine (example 71b): ¹H-NMR (400 MHz, CD₃OD, 298K): 1.42 (d, 3H), 3.15-3.28 (m, 2H), 4.91-5.02 (m, 1H), 8.12-8.18 (m, 2H), 8.62 (d, 1H), 8.73 (d, 2H), 8.79 (d, 2H), 9.23 (s, 1H). MS: 280.2 [M+1]⁺.

By following the procedures of Example 72, but by using the appropriate starting materials, the compounds of formula A wherein R is as indicated in Table 4 below may be obtained.

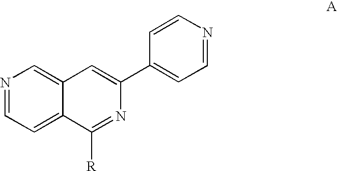

TABLE 4

| | name | R | MS |
|---|---|---|---|
| 73. | (S)-N*1*-(3-Pyridin-4-yl- [2,6]naphthyridin-1-yl)-propane-1,2- diamine | 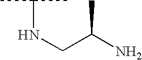 | MH⁺ 280.3 |
| 74. | (S)-N*2*-(3-Pyridin-4-yl- [2,6]naphthyridin-1-yl)-propane-1,2- diamine | 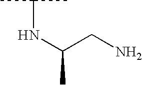 | MH⁺ 280.3 |
| 75. | Rac-(1R,2R)-N-(3-Pyridin-4-yl- [2,6]naphthyridin-1-yl)-cyclohexane-1,2- diamine | 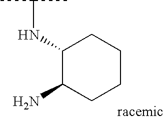 | MH⁺ 320.3 |

By following the procedures of Example 1 (Method A), but by using the appropriate starting materials, i.e. using nicotinic acid methyl ester instead of isonicotinic acid methyl ester, the compounds of formula C wherein R is as indicated in Table 5 below may be obtained.

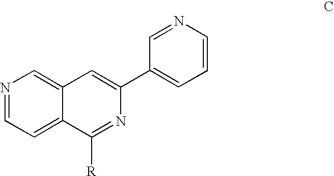

TABLE 5

| | name | R | MS |
|---|---|---|---|
| 76. | N*1*-(3-Pyri- din-3-yl-[2,6]naphthyridin-1- yl)-ethane-1,2-diamine | 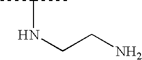 | MH⁺ 266.2 |
| 77. | 2-Methyl-N*1*-(3-pyridin-3-yl- [2,6]naphthyridin-1-yl)-propane-1,2- diamine | 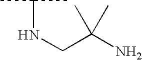 | MH⁺ 294.2 |

### Example 78

### N*1*-[3-(2-Amino-pyridin-4-yl)-[2,6]naphthyridin-1-yl]-2-methyl-propane-1,2-diamine

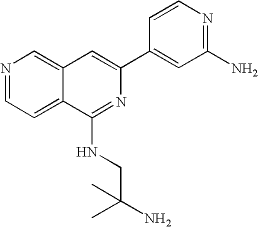

A suspension of N*1*-[3-(2-chloro-pyridin-4-yl)-[2,6]naphthyridin-1-yl]-2-methyl-propane-1,2-diamine (89.8 mg, 0.274 mmol) in toluene (10 mL) is purged with argon for 10 min. Subsequently, benzophenone imine (124 mg, 0.685 mmol), Pd₂(dba)₃ (25.1 mg, 0.0274 mmol), BINAP (17.1 mg, 0.0274 mmol) and NaOtBu (132 mg, 1.37 mmol) are added and the resulting reaction mixture is heated for 18 h at 90° C. under an argon atmosphere. The reaction mixture is filtered over hyflo and the filtrated concentrated in vacuo. The residue is dissolved in a mixture of acetonitrile (1.0 mL) and 0.1% of TFA in water (1.0 mL) and stirred for 2 h at rt. The solvent is removed at reduced pressure and the residue is purified by preparative reverse phase HPLC to afford the title compound as a yellow solid (22.0 mg, 0.071 mmol, 26%, TFA salt). ¹H NMR (400 MHz, DMSO-d₆, 298 K): δ=9.31 (s, 1H), 8.77 (d, J=5.7 Hz, 1H), 8.25 (s, J=5.7 Hz, 1H), 8.11 (bs, 1H), 8.05 (d, J=7.0 Hz, 1H), 7.99 (s, 1H), 7.90 (bs, 2H), 7.78 (s, 1H), 7.60 (d, J=7.0 Hz, 1H), 3.89 (d, J=6.1 Hz, 2H), 1.36 (s, 6H). MS (ES⁺): 309 (M(C₁₇H₂₀N₆)+H)⁺.

### Example 79

### N*1*-[3-(2-Chloro-pyridin-4-yl)-[2,6]naphthyridin-1-yl]-2-methyl-propane-1,2-diamine

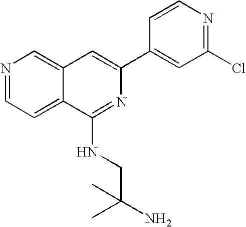

To a solution of a 1:2 mixture of 3-[2-(2-chloro-pyridin-4-yl)-2-oxo-ethyl]-isonicotinonitrile and 3-[2-(2-methoxy-pyridin-4-yl)-2-oxo-ethyl]-isonicotinonitrile (1.5 g, 5.82 mmol) in a mixture of EtOAc/acetic acid 7:3 (58 mL) is added 2-methyl-propane-1,2-diamine (3.08 g, 34.9 mmol). Silica gel 60 (6.98 g) is added and the reaction mixture is stirred for 2 h at rt. The reaction mixture is filtered and the residue washed with EtOAc. The filtrate is concentrated by rotary evaporation and the residue is partitioned between EtOAc and an aqueous 1 M NaOH solution. The organic phase is dried over Na₂SO₄, filtered and concentrated at reduced pressure. Purification by preparative reverse phase HPLC affords the title compound as a yellow powder (256 mg, 0.780 mmol, 13%, TFA salt). ¹H NMR (400 MHz, DMSO-d₆, 298 K): δ=9.27 (s, 1H), 8.72 (d, J=5.4 Hz, 1H), 8.54 (d, J=5.4 Hz, 1H), 8.27 (d, J=5.9 Hz, 1H), 8.22 (s, 1H), 8.21 (d, J=5.9 Hz, 1H), 8.05 (s, 1H), 8.01 (t, J=6.1 Hz, 1H), 7.97 (bs, 2H), 3.91 (d, J=6.1 Hz, 2H), 1.37 (s, 6H). MS (ES⁺): 328 (M(C₁₇H₁₈ClN₅)+H)⁺.

### 3-[2-(2-chloro-pyridin-4-yl)-2-oxo-ethyl]isonicotinonitrile and 3-[2-(2-methoxy-pyridin-4-yl)-2-oxo-ethyl]-isonicotinonitrile

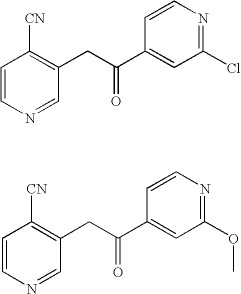

In a three-necked round-bottomed flask equipped with a reflux cooler, 60% NaH in mineral oil (1.35 g, 33.9 mmol) is added to DME (70 mL). The suspension is heated to 95° C. and a solution of 3-methyl-isonicotinonitrile (1.00 g, 8.47 mmol) and 2-chloro-isonicotinic acid methyl ester (2.18 g, 12.71 mmol) in DME (15 mL) is added. The resulting reaction mixture is stirred overnight at 95° C. After cooling down to room temperature the reaction mixture is partitioned between EtOAc and water. The aqueous phase is extracted with EtOAc and the combined organic layers are dried over Na₂SO₄, filtered and concentrated in vacuo to afford a 1:2 mixture of 3-[2-(2-chloro-pyridin-4-yl)-2-oxo-ethyl]-isonicotinonitrile and 3-[2-(2-methoxy-pyridin-4-yl)-2-oxo-ethyl]-isonicotinonitrile as a brown solid (2.25 g, 8.47 mmol, 100%). 3-[2-(2-chloro-pyridin-4-yl)-2-oxo-ethyl]-isonicotinonitrile: MS (ES⁺): 258 (M(C₁₃H₈ClN₃O)+H)⁺; 3-[2-(2-methoxy-pyridin-4-yl)-2-oxo-ethyl]-isonicotinonitrile: MS (ES⁺): 254 (M(C₁₄H₁₁N₃O₂)+H)⁺.

### Example 80

### 4-[1-(2-Amino-2-methyl-propylamino)-[2,6]naphthyridin-3-yl]-1H-pyridin-2-one

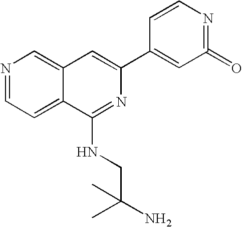

To a solution of N*1*-[3-(2-methoxy-pyridin-4-yl)-[2,6]naphthyridin-1-yl]-2-methyl-propane-1,2-diamine (50.0 mg, 0.154 mmol) in chloroform (5.0 mL) is added at rt iodotrimethylsilane (157.6 mg, 0.77 mmol). The resulting reaction mixture is stirred overnight at 70° C. Methanol (5.0 mL) is added and the reaction mixture is stirred for 24 h at 70° C. After cooling to room temperature the volatiles are removed in vacuo. Purification by FCC (EtOAc/EtOH/25% aqueous NH₃ 10:9:1 affords the title compound as a yellow powder (28 mg, 0.091 mmol, 59%). ¹H NMR (400 MHz, DMSO-d₆, 298 K): δ=9.22 (s, 1H), 8.64 (d, J=5.7 Hz, 1H), 8.26 (d, J=5.7 Hz, 1H), 7.77 (t, J=6.1 Hz, 1H), 7.75 (s, 1H), 7.47 (d, J=7.0 Hz, 1H), 7.27 (bs, 2H), 7.14 (s, 1H), 6.91 (d, J=7.0 Hz, 1H), 3.68 (d, J=6.1 Hz, 2H), 1.76 (s, 6H). MS (ES⁺): 310 (M(C₁₇H₁₉N₅O)+H)⁺.

### N*1*-[3-(2-Methoxy-pyridin-4-yl)-[2,6]naphthyridin-1-yl]-2-methyl-propane-1,2-diamine

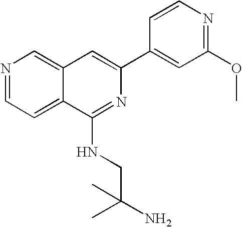

To a solution of a 1:2 mixture of 3-[2-(2-chloro-pyridin-4-yl)-2-oxo-ethyl]-isonicotinonitrile and 3-[2-(2-methoxy-pyridin-4-yl)-2-oxo-ethyl]-isonicotinonitrile (1.5 g, 5.82 mmol) in a mixture of EtOAc/acetic acid 7:3 (58 mL) is added 2-methyl-propane-1,2-diamine (3.08 g, 34.9 mmol). Silica gel 60 (6.98 g) is added and the reaction mixture is stirred for 2 h at rt. The reaction mixture is filtered and the residue washed with EtOAc. The filtrate is concentrated by rotary evaporation and the residue is partitioned between EtOAc and an aqueous 1 M NaOH solution. The organic phase is dried over Na₂SO₄, filtered and concentrated at reduced pressure. Purification by preparative reverse phase HPLC affords the title compound as a yellow powder (600 mg, 1.86 mmol, 32%, TFA salt). ¹H NMR (400 MHz, DMSO-d₆, 298 K): δ=9.27 (s, 1H), 8.69 (d, J=5.6 Hz, 1H), 8.29 (d, J=5.6 Hz, 1H), 8.26 (d, J=5.6 Hz, 1H), 7.97 (bs, 3H), 7.94 (s, 1H), 7.76 (d, J=5.6 Hz, 1H), 7.60 (s, 1H), 3.93 (s, 3H), 3.89 (d, J=6.1 Hz, 2H), 1.35 (s, 6H). MS (ES⁺): 324 (M(C₁₈H₂₁N₅O)+H)⁺.

### Example 81

### 1,1-Dimethyl-2-(3-pyridin-4-yl-[2,6]naphthyridin-1-yloxy)-ethylamine

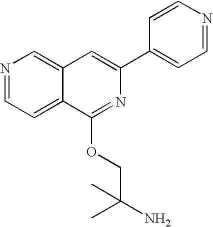

A suspension of [2-(3-pyridin-4-yl-[2,6]naphthyridin-1-yloxy)-ethyl]carbamic acid tert-butyl ester (80.0 mg, 0.218 mmol) in a 4 M solution of HCl in dioxane (3.0 mL) is stirred for 3 h at rt. The reaction mixture is concentrated in vacuo and the residue is crystallized from methanol/diethyl ether to afford the title compound as yellow crystals (48.0 mg, 0.119 mmol, 55%). ¹H NMR (400 MHz, DMSO-d₆, 298 K): δ=9.54 (s, 1H), 9.07 (d, J=5.9 Hz, 2H), 8.93 (d, J=5.4 Hz, 1H), 8.85 (s, 1H), 8.80 (d, J=5.9 Hz, 2H), 8.66-8.55 (m, 3H), 4.71 (s, 2H), 1.51 (s, 6H). MS (ES⁺): 295 (M(C₁₇H₁₈N₄O)+H)⁺.

### [1,1-Dimethyl-2-(3-pyridin-4-yl-[2,6]naphthyridin-1-yloxy)-ethyl]-carbamic acid tert-butyl ester

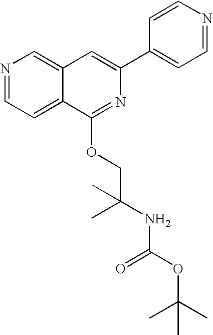

To a solution of (2-hydroxy-1,1-dimethyl-ethyl)-carbamic acid tert-butyl ester (80.6 mg, 0.50 mmol) in DMF (5.0 mL) is added 60% NaH in mineral oil (30.0 mg, 0.75 mmol). The reaction mixture is stirred for 20 min. at 75° C. After cooling to room temperature, 1-bromo-3-pyridin-4-yl-[2,6]naphthyridine (150 mg, 0.525 mmol), Pd₂(dba)₃ (13.7 mg, 0.015 mmol), and BINAP (12.5 mg, 0.020 mmol) are added and the resulting reaction mixture is heated for 2 h at 75° C. under an argon atmosphere. The reaction mixture is cooled to room temperature, filtered over hyflo and the filtrate concentrated in vacuo. The residue is purified by FCC (silica gel, cyclohexane/EtOAc, 1:1→2:8) to afford the title compound as a yellow powder (85.0 mg, 0.232 mmol, 46%). ¹H NMR (400 MHz, DMSO-d₆, 298 K): δ=9.43 (s, 1H), 8.79 (d, J=5.6 Hz, 1H), 8.75 (d, J=6.0 Hz, 2H), 8.40 (s, 1H), 8.17 (d, J=6.0 Hz, 2H), 8.11 (d, J=5.6 Hz, 2H), 7.69 (bs, 1H), 6.83 (bs, 1H), 4.68 (s, 2H), 1.42 (s, 6H), 1.29 (s, 9H). MS (ES⁺): 395 (M(C₂₂H₂₆N₄O₃)+H)⁺.

By following the procedures of Example 81, but by using the appropriate starting materials, the compounds of formula A wherein R is as indicated in Table 6 below may be obtained.

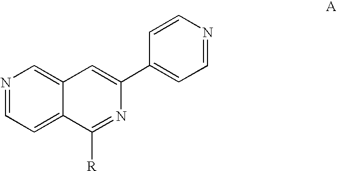

TABLE 6

| | name | R | MS |
|---|---|---|---|
| 82. | 2-(3-Pyri- din-4-yl-[2,6]naphthyridin-1- yloxy)-ethylamine | 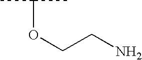 | MH⁺ 267.3 |

### Example 83

### 2-Methyl-N*1*-(7-pyridin-4-yl-isoquinolin-5-yl)-propane-1,2-diamine

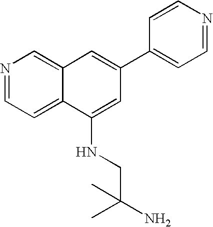

To a solution of 5-bromo-7-pyridin-4-yl-isoquinoline (100.0 mg, 0.351 mmol) in toluene (4.0 mL) is added 1,2-diamino-2-methylpropane (77.3 mg, 0.878 mmol), Pd₂(dba)₃ (32.1 mg, 0.0351 mmol), BINAP (21.8 mg, 0.0351 mmol) and NaOtBu (115 mg, 1.19 mmol). The reaction mixture is purged with argon for 10 min. and subsequently heated for 15 h at 90° C. under an argon atmosphere. After cooling to room temperature, the reaction mixture is diluted with diethyl ether and filtered through a glass filter. The filtrate is concentrated in vacuo and the residue is purified by preparative reverse phase HPLC to afford the title compound as a dark yellow solid (72.3 mg, 0.114 mmol, 32%, TFA salt). ¹H NMR (400 MHz, DMSO-d₆, 298 K): δ=9.44 (s, 1H), 8.84 (d, J=6.2 Hz, 2H), 8.61 (d, J=6.2 Hz, 1H), 8.36 (d, J=6.2 Hz, 1H), 8.10 (d, J=6.2 Hz, 2H) 8.03-7.97 (bs, 3H), 7.38 (s, 1H), 6.72 (t, J=6.1 Hz, 1H), 3.61 (d, J=6.1 Hz, 2H), 1.39 (s, 6H). MS (ES⁺): 293 (M(C₁₈H₂₀N₄)+H)⁺.

### 5-Bromo-7-pyridin-4-yl-isoquinoline

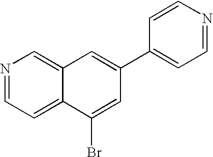

To a solution of 7-pyridin-4-yl-isoquinoline (1.50 g, 7.27 mmol) in 95% H₂SO₄ (15.0 mL) is added portion wise at 0° C. NBS (1.29 g, 7.27 mmol). The reaction mixture is stirred for 6 h at rt, poured onto ice, carefully neutralized (pH=8) with a saturated aqueous NaHCO₃ solution and extracted with EtOAc. The organic layer is dried over Na₂SO₄, filtered and concentrated at reduced pressure to three quarters of its volume. Diethyl ether is added and the precipitated product is filtered off, washed with diethyl ether and dried under high vacuum to give a beige crystalline solid (1.49 g, 5.23 mmol, 72%). ¹H NMR (400 MHz, CDCl₃, 298 K): δ=9.43 (s, 1H), 8.69 (d, J=5.4 Hz, 2H), 8.65 (d, J=6.2 Hz, 1H), 8.50 (s, 1H), 8.37 (s, 1H), 7.88 (d, J=6.2 Hz, 1H), 7.70 (d, J=5.4 Hz, 2H). MS (ES⁺): 285 (M(C₁₄H₉Br⁷⁹N₂)+H)⁺.

### 7-Pyridin-4-yl-isoquinoline

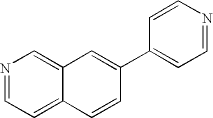

To a solution of 7-bromo-isoquinoline (1.70 g, 8.17 mmol) in DMF (35 mL) are added 4-pyridinyl boronic acid (1.21 g, 9.80 mmol), PdCl₂(PPh₃)₂ (573 mg, 0.812 mmol), and a 2M aqueous Na₂CO₃ solution (24.5 mL). The resulting reaction mixture is stirred for 2.5 h at 100° C. under and argon atmosphere. After cooling to rt, the reaction mixture is filtered through hyflo and the filtrate partitioned between EtOAc and water. The layers are separated and the aqueous layer is extracted with EtOAc. The combined organic layers are washed with water, dried over Na₂SO₄, filtered, and concentrated at reduced pressure. The crude product is purified by FCC (silica gel, EtOAc/EtOH 97:3) to afford the title compound as a yellow oil (1.58 g, 7.66 mmol, 94%). ¹H NMR (400 MHz, DMSO-d₆, 298K) δ=9.40 (s, 1H), 8.69 (d, J=6.1 Hz, 2H), 8.61 (s, 1H), 8.54 (d, J=5.7 Hz, 1H), 8.21 (dd, J=8.6, 1.9 Hz, 1H), 8.10 (d, J=8.6 Hz, 1H) 7.89-7.86 (m, 3H). MS (ES⁺): 207 (M(C₁₄H₁₀N₂)+H)⁺.

By following the procedures of Example 83, but by using the appropriate starting materials, the compounds of formula D wherein R is as indicated in Table 7 below may be obtained.

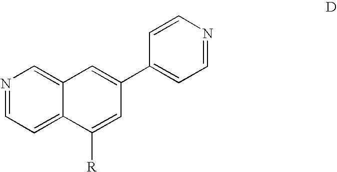

TABLE 7

| | name | R | MS |
|---|---|---|---|
| 84. | N*1*-(7-Pyri- din-4-yl-isoquinolin-5-yl)- ethane-1,2-diamine | 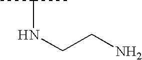 | MH⁺ 265.1 |
| 85. | (S)-N*1*-(7-Pyri- din-4-yl-isoquinolin-5- yl)-propane-1,2-diamine | 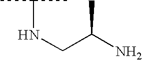 | MH⁺ 279.2 |
| 86. | 5-Piperazin-1-yl-7-pyridin-4-yl- isoquinoline | 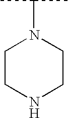 | MH⁺ 291.2 |

### Example 87

### 7-Pyridin-4-yl-isoquinolin-5-ylamine

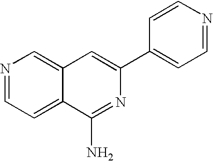

A solution of (3,4-Dimethoxy-benzyl)-(7-pyridin-4-yl-isoquinolin-5-yl)-amine (592.7 mg, 1.59 mmol) in CH₂Cl₂ (10 mL), thioanisol (5.61 mL) and TFA (30 mL) is stirred for 16 h at rt. The reaction mixture is concentrated under reduced pressure, to the residue is added methanol and the formed precipitate is filtered off. The precipitate is dissolved in HCl/MeOH (15 mL, 1.25 M) and is stirred 1 h at rt. The volatiles are evaporated under vacuum to yield the title compound (389 mg, 1.17 mmol, 74%, 3HCl salt) as a yellow solid. MS: 223.1 [M+1]⁺

### (3,4-Dimethyloxy-benzyl)-(3-pyridin-4-yl-[2,6]naphthyridin-1-yl)-amine

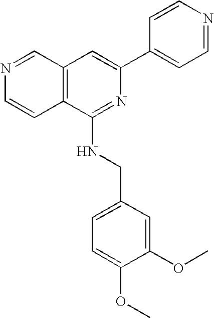

To a solution of 1-chloro-3-pyridin-4-yl-[2,6]naphthyridine (446.6 mg, 1.84 mmol) in 1-Methyl-pyrrolidin-2-one (8 mL) is added 3,4-Dimethoxy-benzylamine (0.835 mL, 5.54 mmol). The reaction mixture is heated to 100° C. for 16 h, cooled to rt, the insoluble impurities are filtered and the filtrate concentrated under vacuum. Methanol is added and the formed precipitated filtered and dried under vacuum to yield to the title compound as a yellow solid (597 mg, 1.6 mmol, 87%), which is used without further purification for the next step. MS: 373.1 [M+1]+

### Example 88

### 3-Bromo-N*1*-(7-pyridin-4-yl-isoquinolin-5-yl)-propane-1,2-diamine

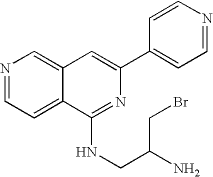

To a solution of 1-chloro-3-pyridin-4-yl-[2,6]naphthyridine (108 mg, 0.447 mmol) in 1-Methyl-pyrrolidin-2-one (1.5 mL) is added azetidin-3-yl-carbamic acid benzyl ester (276 mg, 1.34 mmol). The reaction mixture is heated to 90° C. for 16 h, cooled to rt and diluted with ethyl acetate. The organic layer is washed with NaHCO₃, brine, dried over MgSO₄, filtered and concentrated. Addition of ethyl acetate affords a precipitated that is filtered off. The precipitate is dissolved in acetic acid and HBr 33% in acetic acid (1 mL) is added at rt. The mixture is stirred 15 min. at rt. Diethyl ether is added to the reaction mixture and the formed precipitate filtered off and dried under vacuum to yield to the title compound as a orange solid (142.6 mg, 0.23 mmol, 53%, 3HBr salt). MS: (358.17 and 360.2.1) [M+1]⁺

### Example 89

### N-[1,1-Dimethyl-2-(3-pyridin-4-yl-[2,6]naphthyridin-1-ylamino)-ethyl]-acetamide

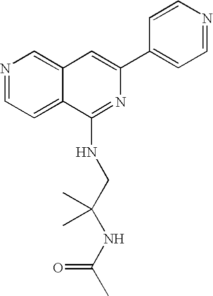

To a solution of 2-methyl-N*1*-(3-pyridin-4-yl-[2,6]naphthyridin-1-yl)-propane-1,2-diamine (99.7 mg, 0.340 mmol) in dichloromethane (2 mL) is added at 0° C. pyridine (26.9 mg, 0.340 mmol) and acetylchlorine (26.7 mg, 0.340 mmol). The resulting dark red suspension is stirred for 1 h at 0° C. and 3 h at rt. The reaction mixture is partitioned between EtOAc and water, the layers are separated and the aqueous layer is extracted with EtOAc. The combined organic layers are washed with water, dried over Na₂SO₄, filtered, and concentrated at reduced pressure. The crude product is purified by FCC (silica gel, EtOAc/EtOH/25% aqueous NH₃ 90:9:1) to afford the title compound as a yellow crystals (61.0 mg, 0.182 mmol, 54%). MS (ES⁺): 336.2 (M(C₁₉H₂₁N₅O)+H)⁺.

### Biological/Pharmacological Part

The compounds of the invention, i.e. of formulae (I), in free form or in pharmaceutically acceptable salt form, exhibit valuable pharmacological properties, e.g. inhibiting Protein Kinase C (PKC), e.g. PKC isotypes like α, β, δ, ε, η or Θ, in particular the isotypes δ, ε, η, and θ and more specifically the isotypes ε and η, inhibiting T-cell activation, proliferation, and, lymphocyte trafficking as indicated in vitro and in vivo tests and are therefore indicated for therapy.

### A. In Vitro

### 1. Protein Kinase C Assay

The compounds of the invention are tested for their activity on different PKC isotypes according to the following method. Assay is performed in a white with clear bottom 384-well microtiterplate with non-binding surface. The reaction mixture (25 μl) contains 1.5 μM of a tridecapeptide acceptor substrate that mimics the pseudo substrate sequence of PKC a with the Ala→Ser replacement, 10 μM ³³P-ATP, 10 mM Mg(NO₃)₂, 0.2 mM CaCl₂, PKC at a protein concentration varying from 25 to 400 ng/ml (depending on the isotype used), lipid vesicles (containing 30 mol % phosphatidylserine, 5 mol % DAG and 65 mol % phosphatidylcholine) at a final lipid concentration of 0.5 mM, in 20 mM Tris-HCl buffer pH 7.4+0.1% BSA. Incubation is performed for 60 min at room temperature. Reaction is stopped by adding 50 μl of stop mix (100 mM EDTA, 200 μM ATP, 0.1% Triton X-100, 0.375 mg/well streptavidin-coated SPA beads in phosphate buffered saline w/o Ca, Mg. After 10 min incubation at room temperature, the suspension is spun down for 10 min at 300 g. Incorporated radioactivity is measured in a Trilux counter for 1 min. IC₅₀ measurement is performed on a routine basis by incubating a serial dilution of inhibitor at concentrations ranging between 1-1000 nM. IC₅₀ values are calculated from the graph by curve fitting with XL Fit® software.

### 2. Protein Kinase C θ Assay

Human recombinant PKCθ is used under the assay conditions as described above. In this assay, compounds of the invention inhibit PKC θ with an IC₅₀≦1 μM.

### 3. Protein Kinase Cα Assay

Human recombinant PKCα was obtained from Oxford Biomedical Research and is used under the assay conditions as described under Section A.1 above. In this assay, compounds of the invention inhibit PKCα with an IC₅₀≦1 μM.

### 4. Protein Kinase Cβ1 Assay

Human recombinant PKCβ1 was obtained from Oxford Biomedical Research and is used under the assay conditions as described under Section A.1 above. In this assay, compounds of the invention inhibit PKCβ1 with an IC₅₀≦1 μM.

### 5. Protein Kinase Cδ Assay

Human recombinant PKC was obtained from Oxford Biomedical Research and is used under the assay conditions as described under Section A.1 above. In this assay, compounds of the invention inhibit PKCδ with an IC₅₀≦1 μM.

### 6. Protein Kinase Cε Assay

Human recombinant PKCη was obtained from Oxford Biomedical Research and is used under the assay conditions as described under Section A.1 above. In this assay, compounds of formula of the invention inhibit PKCε with an IC₅₀≦1 μM.

### 7. Protein Kinase Cθ Assay

Human recombinant PKCη was obtained from PanVera and is used under the assay conditions as described under Section A.1 above. In this assay, compounds of the invention inhibit PKCη with an IC₅₀≦1 μM.

### 8. PKD-1 Assay

The assay to measure protein kinase D1 (PKD1) activity is a time-resolved fluorescence resonance transfer (TR-FRET) assay using PerkinElmer's LANCE™ technology. In this case, a biotinylated syntide-2 peptide is used as the substrate in this reaction. Phosphorylation of the syntide-2 substrate is detected by a specific antibody that recognizes the phosphorylated peptide. A second fluorophore, APC, is conjugated to streptavidin that binds the biotinylated syntide-2 peptide. For detection, the europium fluorophore can be excited by 340 nM light which then emits at 615 nM. Therefore, when the europium labeled secondary antibody binds on the phosphorylated peptide, it is brought into close contact with the APC and excites this fluorophore. The APC emission is at 665 nM and the 665 nM:615 nM ratio is a readout of PKD1 activity.

This assay is performed with full length wild-type enzyme that is expressed and purified from Sf9 insect cells. The reaction buffer consists of 35 mM Tris-HCl pH 7.5, 5 mM MgCl₂, 0.02% Tween-20, 20 μM ATP, 1 mM DTT and 0.2 μg/mL PKD1 enzyme. The enzyme reaction is initiated by the addition of 2 μM syntide-2 peptide substrate and the reaction carried out for 50 minutes at room temperature. The reaction is stopped by a stop/detection buffer consisting of 50 mM EDTA, 0.18 mg/mL rabbit polyclonal anti-phospho syntide-2 antibody, 0.5 nM europium labeled anti-rabbit IgG and 10 nM streptavidin conjugated APC. After a one hour incubation with the stop/detection buffer, the reaction is read on an Envision 2100 Reader using a LANCE™ Eu/APC dual protocol. As described above, a 665 nM:615 nM ratio is determined to measure substrate phosphorylation and enzyme activity. Compounds are typically tested in an 11 point dose response fashion in triplicate for each concentration used. IC₅₀ values are calculated using an Activity Base (IDBS) software program.

### 9. PKN-2 Assay

The assay is performed using the Upstate IC₅₀ Profiler Express™ service. In a final reaction volume of 25 mL, human recombinant PKN-2 (5-10 mU) is incubated with 50 mM Tris pH 7.5, 0.1 mM EGTA, 0.1% β-mercaptoethanol, 30 μM undecapeptide (AKRRRLSSLRA), 10 mM magnesium acetate and γ-³³P-ATP (specific activity approx. 500 cpm/pmol, concentration as required). The reaction is initiated by the addition of the Mg/ATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

### 10. ROCK-II Assay

The assay is performed using the Upstate IC₅₀ Profiler Express™ service. In a final reaction volume of 25 mL, human recombinant ROCK-II (5-10 mU) is incubated with 50 mM Tris pH 7.5, 0.1 mM EGTA, 30 μM KEAKEKRQEQIAKRRRLSSLRASTSKSGGSQK, 10 mM magnesium acetate and γ-³³P-ATP (specific activity approx. 500 cpm/pmol, concentration as required). The reaction is initiated by the addition of the Mg/ATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

### 11. Allogeneic Mixed Lymphocyte Reaction (MLR)

The two-way MLR is performed according to standard procedures (J. Immunol. Methods, 1973, 2, 279 and Meo T. et al., Immunological Methods, New York, Academic Press, 1979, 227-39). Briefly, spleen cells from CBA and BALB/c mice (1.6×10⁵ cells from each strain per well in flat bottom tissue culture microtiter plates, 3.2×10⁵ in total) are incubated in RPMI medium containing 10% FCS, 100 U/ml penicillin, 100 μg/ml streptomycin (Gibco BRL, Basel, Switzerland), 50 μM 2-mercaptoethanol (Fluka, Buchs, Switzerland) and serially diluted compounds. Seven three-fold dilution steps in duplicates per test compound are performed. After four days of incubation 1 μCi ³H-thymidine is added. Cells are harvested after an additional five-hour incubation period, and incorporated ³H-thymidine is determined according to standard procedures. Background values (low control) of the MLR are the proliferation of BALB/c cells alone. Low controls are subtracted from all values. High controls without any sample are taken as 100% proliferation. Percent inhibition by the samples is calculated, and the concentrations required for 50% inhibition (IC₅₀ values) are determined.

### 12. Bone Marrow Cell Proliferation (BM) Assay

Bone marrow cells from CBA mice (2.5×10⁴ cells per well in flat bottom tissue culture microtiter plates) are incubated in 100 μL RPMI medium containing 10% FCS, 100 U/mL penicillin, 100 μg/mL streptomycin (Gibco BRL, Baselm Switzerland), 50 μM 2-mercaptoethanol (Fluka, Buchs, Switzerland), WEHI-3 conditioned medium (7.5% v/v) and L929 conditioned medium (3% v/v) as a source of growth factors and serially diluted compounds. Seven three-fold dilution steps in duplicates per test compounds are performed. After four days of incubation 1 μCi ³H-thymidine is added. Cells are harvested after an additional five-hour incubation period, and incorporated ³H-thymidine is determined according to standard procedures. Conditioned media are prepared as follows. WEHI-3 cells (ATCC TIB68) and L929 cells (ATCC CCL 1) are grown in RPMI medium until confluence for 4 days and one week, respectively. Cells are harvested, resuspended in the same culture flasks in medium C containing 1% FCS (Schreier and Tess 1981) for WEHI-3 cells and RPMI medium for L929 cells and incubated for 2 days (WEHI-3) or one week (L929). The supernatant is collected, filtered through 0.2 μm and stored in aliquots at −80° C. Cultures without test compounds and without WEHI-3 and L929 supernatants are used as low control values. Low control values are substracted from all values. High controls without any sample are taken as 100% proliferation. Percent inhibition by the samples is calculated and the concentrations required for 50% inhibition (IC₅₀ values) are determined.

### Results

The assays used are described herein above.

The ratios of the IC₅₀ value for PKCα to the IC₅₀ value for PKCη, of the IC₅₀ value for PKC β to the IC₅₀ value for PKCη, of the IC₅₀ value for PKCδ to the IC₅₀ value for PKCη, of the IC₅₀ value for PKCε to the IC₅₀ value for PKCη, of the IC₅₀ value for PKCθ to the IC₅₀ value for PKCη, and the IC₅₀ value for inhibition of in the MLR assay to the IC₅₀ value as determined by the BM assay, obtained for some compounds of the invention are indicated in table 8.

PKC α, β, δ, ε, η and θ assays, MLR and BM assays, are as described hereinabove.

TABLE 8

| Example | α/η | β/η | δ/η | ε/η | θ/η | BM/MLR |
|---|---|---|---|---|---|---|
| (0) | 108 | 105 | 5 | 2 | 16 | 22 |
| (23) | 17 | 14 | 15 | 1 | 26 | 24 |
| (71a) | 506 | 196 | 4 | 3 | 30 | 30 |

The compounds of the invention typically show a selectivity of at least 10 fold, preferably 20 fold, more preferably 100 fold for the PKCs ε and η, and eventually δ and θ, over the classical PKC isotypes α and β.

Selectivity for the ε, η or δ, ε, η, θ isoforms of the classical PKC isotypes can be measured by comparing the IC₅₀ of the compound for the ε, or η PKC or δ, ε, η, θ PKC to the IC₅₀ of the compound for the other PKC isotypes, e.g. α, and β. Typically, the selectivity can be determined by calculating the ratio of IC₅₀ of the compound for the ε or η PKC isotypes or the δ, ε, η, θ PKC isotypes to the IC₅₀ of the compound for the α or β PKC.

IC₅₀ values may be obtained, for example, according to the hereineabove mentioned PKC assay(s).

The preferred compounds of the invention typically have an IC₅₀ value for the ε and η, in addition depending on the chemical variation also efficacy in PKC δ and θ, PKCs of ≦1 μM, preferably ≦100 nM in the hereinabove mentioned assay(s). For example, compound of example 81 inhibits PKCη with an IC₅₀ of 162 nM; compound of example 83 with an IC₅₀ of 54 nM.

### B. In Vivo

### Peripheral Lymphocyte Reduction

Rats are subjected to a single oral dose of either placebo (control) or compound at different doses. Sublingual blood for hematological monitoring is collected before compound administration (baseline) and 2, 6, 8, and 24 hours after compound application. To this end, rats are anaesthetized with isoflurane and whole blood (<200 μl) is sampled from the sublingual vein in EDTA-coated Eppendorf tubes. Subsequently, whole blood is subjected to hematological analysis using an automated hematology analyzer for counting different blood cell types and measuring various blood components. This includes red blood cells, hemoglobin, hematocrit, platelets and white blood cells such as neutrophils, lymphocytes, monocytes, eosinophils and basophils.

### Localized Graft-Versus-Host Model (GvH)

Spleen cells (2×10⁷) from Wistar/F rats are injected subcutaneously into the right hind footpad of (Wistar/F×Fischer 344) F₁ hybrid rats. The left footpad is left untreated. The animals are treated with the test compounds on 4 consecutive days (0-3). The popliteal lymph nodes are removed on day 7, and the weight differences between two corresponding lymph nodes are determined. The results are expressed as the inhibition of lymph node enlargement (given in percent) comparing the lymph node weight differences in the experimental groups to the weight difference between the corresponding lymph nodes from a group of animals left untreated with a test compound.

### Rat Heart Transplantation

Heterotopic heart allotransplantation using the strain combination BN (RT¹ haplotype, donor) to Male Lewis (RT¹ haplotype, recepient) is performed according to standard transplantation procedure as e.g. described in WO2002038561. Graft function is monitored by daily palpation of the beating donor heart through the abdominal wall. Rejection is considered to be complete when heart beat stops. Prolongation of graft survival is obtained in animals treated with a compound of the present invention administered orally at a daily dose of 1 to 100 mg/kg bid.

### Results

The compounds of the invention typically induce a rapid and transient reduction of the number of peripheral lymphocytes after a single administration of the compound. As seen in Table 9, the number of peripheral lymphocytes drops within 2 hours after compound administration to 36% of the original counts, and then return to normal numbers by 24 hours after treatment. In the localized GvH model the compounds inhibit lymph node swelling by >90%. The preferred compounds of the invention typically are efficacious at daily oral doses of 30 mg/kg.

TABLE 9

Lymphocyte count (% of control)

| Time (hours) | Placebo | Compound of Example No. 1 |
|---|---|---|
| 0 | 100 (+/−0) | 100 (+/−0) |
| 2 | 78 (+/−7) | 36 (+/−1) |
| 4 | 92 (+/−4) | 36 (+/−4) |
| 8 | 78 (+/−10) | 63 (+/−6) |
| 24 | 80 (+/−8) | 104 (+/−11) |

Definition of Placebo: PEG400/5% glucose
Compound of Example No. 1 is dissolved in PEG400/5% glucose at a concentration of 6 mg/ml

### Utility Statement

The compounds of the present invention are typically useful in the prevention or treatment of disorders or diseases where PKC, PKD, PKN-1/2, CDK-9, MRCK-beta, PASK, PRKX, ROCK-I/II or mediators of other kinases play a role, e.g. diseases or disorders mediated by T lymphocytes, B lymphocytes, mast cells, eosinophils or cardiomyocytes e.g. acute or chronic rejection of organ or tissue allo- or xenografts, graft-versus-host disease, host-versus-graft disease, atheriosclerosis, cerebral infarction, vascular occlusion due to vascular injury such as angioplasty, restenosis, fibrosis (especially pulmonary, but also other types of fibrosis, such as renal fibrosis), angiogenesis, hypertension, heart failure, chronic obstructive pulmonary disease, CNS disease such as Alzheimer disease or amyotrophic lateral sclerosis, cancer, infectious disease such as AIDS, septic shock or adult respiratory distress syndrome, ischemia/reperfusion injury e.g. myocardial infarction, stroke, gut ischemia, renal failure or hermorrhage shock, or traumatic shock. The compounds of the invention are also useful in the treatment and/or prevention of acute or chronic inflammatory diseases or disorders or autoimmune diseases e.g. sarcoidosis, fibroid lung, idiopathic interstitial pneumonia, obstructive airways disease, including conditions such as asthma, intrinsic asthma, extrinsic asthma, dust asthma, particularly chronic or inveterate asthma (for example late asthma and airway hyperreponsiveness), bronchitis, including bronchial asthma, infantile asthma, rheumatoid arthritis, osteoarthritis, systemic lupus erythematosus, nephrotic syndrome lupus, Hashimoto's thyroiditis, multiple sclerosis, myasthenia gravis, type I diabetes mellitus and complications associated therewith, type II adult onset diabetes mellitus, uveitis, nephrotic syndrome, steroid dependent and steroid-resistant nephrosis, palmoplantar pustulosis, allergic encephalomyelitis, glomerulonephritis, psoriasis, psoriatic arthritis, atopic eczema (atopic dermatitis), allergic contact dermatitis, irritant contact dermatitis and further eczematous dermatitises, seborrheic dermatitis, lichen planus, pemphigus, bullous pemphigoid, epidermolysis bullosa, urticaria, angioedemas, vasculitides, erythemas, cutaneous eosinophilias, acne, alopecia greata, eosinophilic fasciitis, atherosclerosis, conjunctivitis, keratoconjunctivitis, keratitis, vernal conjunctivitis, uveitis associated with Behcet's disease, herpetic keratitis, conical cornea, Sjoegren's syndrome, dystorphia epithelialis corneae, keratoleukoma, ocular pemphigus, Mooren's ulcer, scleritis, Graves' opthalmopathy, severe intraocular inflammation, inflammation of mucosa or blood vessels such as leukotriene B4-mediated diseases, gastric ulcers, vascular damage caused by ischemic diseases and thrombosis, cardiac hypertrophy, ischemic bowel disease, inflammatory bowel disease (e.g. Crohn's disease or ulcerative colitis), necrotizing enterocolitis, renal diseases including interstitial nephritis, Goodpasture's syndrome hemolytic uremic syndrome and diabetic nephropathy, nervous diseases selected from multiple myositis, Guillain-Barre syndrome, Meniere's disease and radiculopathy, collagen disease including scleroderma, Wegener's granuloma and Sjogren' syndrome, chronic autoimmune liver diseases including autoimmune hepatitis, primary biliary cirrhosis and sclerosing cholangitis), partial liver resection, acute liver necrosis (e.g. necrosis caused by toxins, viral hepatitis, shock or anoxia), cirrhosis, fulminant hepatitis, pustular psoriasis, Behcet's disease, active chronic hepatitis, Evans syndrome, pollinosis, idiopathic hypoparathyroidism, Addison disease, autoimmune atrophic gastritis, lupoid hepatitis, tubulointerstitial nephritis, membranous nephritis, or rheumatic fever. The compounds of formula I are useful for treating tumors, e.g. breast cancer, genitourinary cancer, lung cancer, gastrointestinal cancer, epidermoid cancer, melanoma, ovarian cancer, pancreas cancer, neuroblastoma, head and/or neck cancer or bladder cancer, or in a broader sense renal, brain or gastric cancer; in particular (i) a breast tumor; an epidermoid tumor, such as an epidermoid head and/or neck tumor or a mouth tumor; a lung tumor, for example a small cell or non-small cell lung tumor; a gastrointestinal tumor, for example, a colorectal tumor; or a genitourinary tumor, for example, a prostate tumor (especially a hormone-refractory prostate tumor); or (ii) a proliferative disease that is refractory to the treatment with other chemothe-rapeutics; or (iii) a tumor that is refractory to treatment with other chemotherapeutics due to multidrug resistance. They are also useful for treating tumors of blood and lymphatic system (e.g. Hodgkin's disease, Non-Hodgkin's lymphoma, Burkitt's lymphoma, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma). Myeloid cancer includes e.g. acute or chronic myeloid leukaemia.

Where a tumor, a tumor disease, a carcinoma or a cancer are mentioned, also metastasis in the original organ or tissue and/or in any other location are implied alternatively or in addition, whatever the location of the tumor and/or metastasis.

Preferably the compounds of the present invention are in particular useful in the prevention and/or treatment of a disease or a disorder mediated by T lymphocytes such as acute or chronic rejection of organ or tissue allo- or xenografts, graft-versus-host disease, host-versus-graft disease, multiple sclerosis, psoriasis, or rheumatoid arthritis.

In another aspect the present invention describes compounds having a preferred inhibitory efficacy (IC₅₀) of at least 100 nanomolar for both the kinases PKCₑₜₐ and PKN-1/2 as determined by the assays as described hereinbefore in the manufacture of a medicament for treating an autoimmune disorder, and in particular in the prevention and treatment of acute or chronic rejection of organ or tissue allo- or xenografts, graft-versus-host disease, and host-versus-graft disease.

For the above uses the required dosage will of course vary depending on the mode of administration, the particular condition to be treated and the effect desired. In general, satisfactory results are indicated to be obtained systemically at daily dosages of from about 0.02 to 25 mg/kg per body weight. An indicated daily dosage in the larger mammal, e.g. humans, is in the range from about 0.2 mg to about 2 g, conveniently administered, for example, in divided doses up to four times a day or in retard form. Suitable unit dosage forms for oral administration comprise from ca.0.1 to 500 mg active ingredient.

The compounds of the invention may be administered by any conventional route, in particular parenterally, for example in the form of injectable solutions or suspensions, enterally, e.g. orally, for example in the form of tablets or capsules, topically, e.g. in the form of lotions, gels, ointments or creams, or in a nasal or a suppository form. Topical administration is e.g. to the skin. A further form of topical administration is to the eye. Pharmaceutical compositions comprising a compound of the invention in association with at least one pharmaceutical acceptable carrier or diluent may be manufactured in conventional manner by mixing with a pharmaceutically acceptable carrier or diluent.

The compounds of the invention may be administered in free form or in pharmaceutically acceptable salt form, e.g. as indicated above. Such salts may be prepared in conventional manner and exhibit the same order of activity as the free compounds.

In accordance with the foregoing, the present invention also provides:

(1) A compound of formula I or a pharmaceutically acceptable salt thereof, for use as a pharmaceutical;
(2) A compound of formula I or a pharmaceutically acceptable salt thereof, for use as a PKC inhibitor, for example for use in any of the particular indications hereinbefore set forth;
(3) A pharmaceutical composition, e.g. for use in any of the indications herein before set forth, comprising a compound of formula I or a pharmaceutically acceptable salt thereof, together with one or more pharmaceutically acceptable diluents or carriers therefor.
(4) A method for the treatment or prevention of a disease or condition in which PKC activation plays a role or is implicated, e.g. for the treatment of any of particular indication hereinbefore set forth in a subject in need thereof which comprises administering to the subject an effective amount of a compound of formula I or a pharmaceutically acceptable salt thereof;
(5) The use of a compound of formula I or a pharmaceutically acceptable salt thereof, for the manufacture of a medicament for the treatment or prevention of a disease or condition in which PKC activation plays a role or is implicated; e.g. as indicated above.

### Combinations

The compounds of the invention may be administered as the sole active ingredient or in conjunction with, e.g. as an adjuvant to, other drugs e.g. in immunosuppressive or immunomodulating regimens or other anti-inflammatory agents, e.g. for the treatment or prevention of allo- or xenograft acute or chronic rejection or inflammatory or autoimmune disorders, a chemotherapeutic agent or an anti-infective agent, e.g. an anti-viral agent such as e.g. an anti-retroviral agent or an antibiotic.

For example, the compounds of the invention may be used in combination with a calcineurin inhibitor, e.g. cyclosporin A, ISA247 or FK 506; a mTOR inhibitor, e.g. rapamycin, 40-O-(2-hydroxyethyl)-rapamycin, CC1779, ABT578, TAFA-93, AP23573, AP23464, AP23841, biolimus-7 or biolimus-9; an ascomycin having immuno-suppressive properties, e.g. ABT-281, ASM981, etc.; corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a S1P receptor agonist or modulator, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts; immunosuppressive monoclonal antibodies, e.g., monoclonal antibodies to leukocyte receptors, e.g., MHC, CD2, CD3, CD4, CD7, CD8, CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists, e.g. natalizumab (ANTEGREN®); or antichemokine antibodies or antichemokine receptor antibodies, or low molecular weight chemokine receptor antagonists, e.g. anti MCP-1 antibodies.

A compound of the invention may also be used in combination with other antiproliferative agents. Such antiproliferative agents include, but are not limited to:

(i) aromatase inhibitors, e.g. steroids, especially exemestane and formestane and, in particular, non-steroids, especially aminoglutethimide, vorozole, fadrozole, anastrozole and, very especially, letrozole;
(ii) antiestrogens, e.g. tamoxifen, fulvestrant, raloxifene and raloxifene hydrochloride;
(iii) topoisomerase I inhibitors, e.g. topotecan, irinotecan, 9-nitrocamptothecin and the macromolecular camptothecin conjugate PNU-166148 (compound A1 in WO99/17804);
(iv) topoisomerase II inhibitors, e.g. the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide;
(v) microtubule active agents, e.g. the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D;
(vi) alkylating agents, e.g. cyclophosphamide, ifosfamide and melphalan;
(vii) histone deacetylase inhibitors;
(viii) farnesyl transferase inhibitors;
(ix) COX-2 inhibitors, e.g. celecoxib (Celebrex®), rofecoxib (Vioxx®) and lumiracoxib (COX189);
(x) MMP inhibitors;
(xi) mTOR inhibitors;
(xii) antineoplastic antimetabolites, e.g. 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719;
(xiii) platin compounds, e.g. carboplatin, cis-platin and oxaliplatin;
(xiv) compounds decreasing the protein kinase activity and further anti-angiogenic compounds, e.g. (i) compounds which decrease the activity of the Vascular Endothelial Growth Factor (VEGF) (b) the Epidermal Growth Factor (EGF), c-Src, protein kinase C, Platelet-derived Growth Factor (PDGF), Bcr-Abl tyrosine kinase, c-kit, Flt-3 and Insulin-like Growth Factor I Receptor (IGF-IR) and Cyclin-dependent kinases (CDKs); (ii) Imatinib, midostaurin, Iressa™ (ZD1839), CGP 75166, vatalanib, ZD6474, GW2016, CHIR-200131, CEP-7055/CEP-5214, CP-547632 and KRN-633; (iii) thalidomide (THALOMID), celecoxib (Celebrex), SU5416 and ZD6126;
(xv) gonadorelin agonists, e.g. abarelix, goserelin and goserelin acetate;
(xvi) anti-androgens, e.g. bicalutamide (CASODEX™);
(xvii) bengamides;
(xviii) bisphosphonates, e.g. etridonic acid, clodronic acid, tiludronic acid, pamidronic acid, alendronic acid, ibandronic acid, risedronic acid and zoledronic acid;
(xix) antiproliferative antibodies, e.g. trastuzumab (Herceptin™), Trastuzumab-DM1, erlotinib (Tarceva™), bevacizumab (Avastin™), rituximab (Rituxan®), PRO64553 (anti-CD40) and 2C4 Antibody;
(xx) temozolomide (TEMODAL®);
(xxi) Statins.

The structure of the active agents identified by code nos., generic or trade names may be taken from the actual edition of the standard compendium “The Merck Index” or from databases, e.g. Patents International (e.g. IMS World Publications).

In accordance with the foregoing the present invention provides in a yet further aspect:

(6) A method as defined above comprising co-administration, e.g. concomitantly or in sequence, of a therapeutically effective amount of a) a compound of formula I or a pharmaceutically acceptable salt thereof, and b) a second drug substance, said second drug substance being for example for use in any of the particular indications hereinbefore set forth.
(7) A combination, e.g. a kit, comprising a therapeutically effective amount of a compound of formula I or a pharmaceutically acceptable salt thereof, and a second drug substance, said second drug substance being for example as disclosed above.

Where a compound of the invention is administered in conjunction with other immunosuppressive/immunomodulatory, anti-inflammatory or antineoplastic agent, e.g. as disclosed above, dosages of the co-administered drug or agent will of course vary depending on the type of co-drug or agent employed, or the specific drug or agent used, or the condition being treated and so forth.

1. A compound of formula I 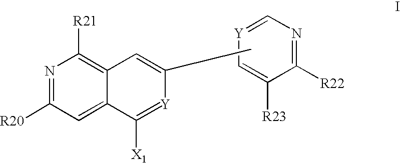
wherein X₁ is a ligand of formula (a), (b), (c), (d), or (e), 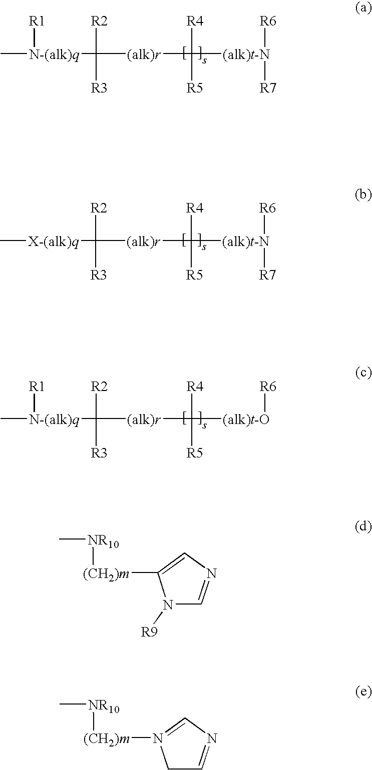
and wherein
X is O or S;
alk is alkylene,
Y and Y₁ are independent from each other and are CH or N,
R₂₀ and R₂₁ are independently selected from the group consisting of hydrogen, cyano, amino, N-alkylamino, —NH-alkylene-aryl, —NH-aryl, halo, alkoxy, hydroxyl, and mercapto;
R₂₂ is hydrogen,
R₂₃ is selected from the group consisting of hydrogen, lower alkyl, halo, hydroxyl, SH, CN and CF₃,
R₁ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl; or R₁ and R₂ are collectively alkyl and form together with the atoms to which they are attached a 4 to 8 membered ring system which may be interrupted by —O—, —S—, —NR₈—, or —CO—, or R₁ and R₄ are collectively alkyl and form together with the atoms to which they are attached a 4 to 8 membered ring system which may be interrupted by —O—, —S—, —NR₈—, or —CO—, or R₁ and R₆ are collectively alkyl and form together with the atoms to which they are attached a 4 to 8 membered ring system which may be interrupted by —O—, —S—, —NR₈—, or —CO—;
R₂ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl; or R₂ and R₃ are collectively alkyl and form together with the atoms to which they are attached a 3 to 7 membered ring system which may be interrupted by —O—, —S—, —NR₈—, or —CO—, or R₂ and R₄ are collectively alkyl and form together with the atoms to which they are attached a 5 to 10 membered ring system which may be interrupted by —O—, —S—, —NR₈—, or —CO—, or R₂ and R₆ are collectively alkyl and form together with the atoms to which they are attached a 5 to 10 membered ring system which may be interrupted by —O—, —S—, —NR₈—, or —CO—;
R₃ is hydrogen; alkyl; alkyl terminated by OH, Oalkyl, NH₂, NHalkyl, N(alkyl)₂, COOK CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl, or R₃ and R₁ are collectively alkyl and form together with the atoms to which they are attached a 5 to 10 membered ring system which may be interrupted by —O—, —S—, —NR₈—, or —CO—, or R₃ and R₄ are collectively alkyl and form together with the atoms to which they are attached a 5 to 10 membered ring system which may be interrupted by —O—, —S—, —NR₈—, or —CO—, or R₃ and R₄ are collectively alkyl and form together with the atoms to which they are attached a 5 to 10 membered ring system which may be interrupted by —O—, —S—, —NR₈—, or —CO—, or R₃ and R₂ combine together to one oxygen atom of a carbonyl-group;
R₄ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl; or R₄ and R₅ are collectively alkyl and form together with the atoms to which they are attached a 3 to 7 membered ring system which may be interrupted by —O—, —S—, —NR₈—, or —CO—, or R₄ and R₅ are collectively alkyl and form together with the atoms to which they are attached a 4 to 8 membered ring system which may be interrupted by —O—, —S—, —NR₈—, or —CO—, or R₄ and R₅ combine together to one oxygen atom of a carbonyl-group;
R₅ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; aryl(lower)alkyl; or aryl; or R₅ and R₆ are collectively alkyl and form together with the atoms to which they are attached a 4 to 8 membered ring system which may be interrupted by —O—, —S—, —NR₈—, or —CO—, or R₅ and R₃ are collectively alkyl and form together with the atoms to which they are attached a 4 to 8 membered ring system which may be interrupted by —O—, —S—, —NR₈—, or —CO—, or R₅ and R₆ are collectively alkyl and form together with the atoms to which they are attached a 4 to 8 membered ring system which may be interrupted by —O—, —S—, —NR₈—, or —CO—;
R₆ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl; or R₆ and R₇ are collectively alkyl and form together with the atoms to which they are attached a 3 to 8 membered ring system which may be interrupted by —O—, —S—, —NR₈—, or —CO—;
R₇ is hydrogen; alkyl; alkyl terminated by OH, Oalkyl, NH₂, NHalkyl, N(alkyl)₂, COOH, CONH₂, CONHalkyl or CON(alkyl)₂; alkyl interrupted by O, S, C═O, CONH, CONalkyl, NHCO, NalkylCO, NH, or N-alkyl; or aryl(lower)alkyl; or R₇ and R₅ are collectively alkyl and form together with the atoms to which they are attached a 5 to 10 membered ring system which may be interrupted by —O—, —S—, —NR₈—, or —CO—, or R₇ and R₃ are collectively alkyl and form together with the atoms to which they are attached a 5 to 10 membered ring system which may be interrupted by —O—, —S—, —NR₈—, or —CO—;
R₉ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl;
R₁₀ is hydrogen; alkyl; alkyl terminated by OH, Oalkyl, NH₂, NHalkyl, N(alkyl)₂, COOH, CONH₂, CONHalkyl, or CON(alkyl)₂; alkyl interrupted by O, S, C═O, CONH, CONalkyl, NHCO, NalkylCO, NH, or N-alkyl; or aryl(lower)alkyl;
m is an integer and is from 1-8,
q, r, s, and t are independent from each other and are 0 or 1; or a pharmaceutically acceptable salt thereof.

2. The compound of claim 1 wherein Y is N; or a pharmaceutically acceptable salt thereof.

3. The compound of claim 1, wherein Y₁═N, the pyrimidine ring in formula (I) is a 4-pyrimidyl substituent, in accordance to formula (IIa); or
wherein Y₁═CH, the pyridyl substituent in formula (I) is a 4-pyridyl substituent, in accordance to formula (IIb): 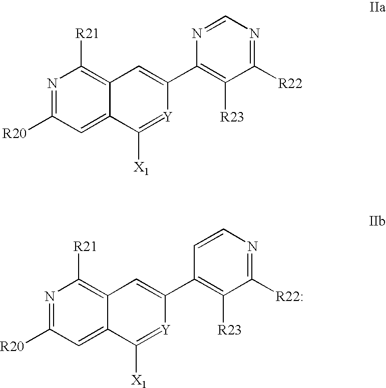
or a pharmaceutically acceptable salt thereof.

4. The compound in accordance to claim 3, wherein Y is N; or a pharmaceutically acceptable salt thereof.

5. The compound in accordance to claim 3 and 4, wherein Y═N and has formula III: 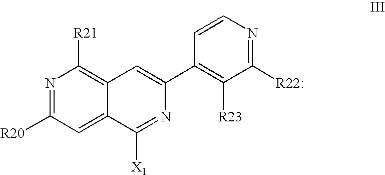
or a pharmaceutically acceptable salt thereof.

6. The compound in accordance to claim 1, wherein ligand X₁ is selected from a residue in accordance to formula (a), (b) and (c); or a pharmaceutically acceptable salt thereof.

7. The compound in accordance to claim 1, wherein ligand X₁ is selected from the group of formulae (d) and (e); or a pharmaceutically acceptable salt thereof.

8. The compound in accordance to claim 1, wherein:
ligand X₁ is —NR_{B}-alkylene-N(alkyl)₂, —NR₈-alkylene-NH-alkyl, or —NR₈-alkylene-NH₂, wherein R₈ is hydrogen or lower alkyl, wherein alkyl is lower alkyl and wherein alkylene is linear, branched, or cyclic and bonded in any position; or
wherein ligand X₁ is —O-alkylene-N(alkyl)₂, —O-alkylene-NH-alkyl, or —O-alkylene-NH₂, wherein alkyl is lower alkyl and wherein alkylene is linear, branched, or cyclic and bonded in any position; or
wherein ligand X₁ is —NR₈-alkylene-OH or —NR₈-alkylene-O-alkyl, wherein R₈ is hydrogen or lower alkyl, wherein alkyl is lower alkyl and wherein alkylene is linear, branched, or cyclic and bonded in any position or
wherein ligand X₁ is —NR₈-alkylene-NH₂ and R₈ is hydrogen, wherein said alkylene is linear, branched, or cyclic and bonded in any position; or a pharmaceutically acceptable salt thereof.

9. The pH compound in accordance to claim 1, wherein in a ligand of formula (a), (b) or (c) the substituents R₁ to R₆ are independently from each other hydrogen, alkyl, or aryl, and R₇ is hydrogen, alkyl, aryl(lower)alkyl, alkyl-carbonyl, or alkyloxy-carbonyl; or a pharmaceutically acceptable salt thereof.

10. A pharmaceutical composition comprising a compound of formula I in free form or pharmaceutically acceptable salt form in association with a pharmaceutically acceptable diluent or carrier therefore.

11. A pharmaceutical combination, comprising a) a first agent which is a compound of claim 1, in free form or in pharmaceutically acceptable salt form, and b) at least one co-agent, selected from an immunosuppressant, immunomodulatory, anti-inflammatory, chemotherapeutic and anti-infectious agent.

12. (canceled)

13. A method for preventing or treating disorders or diseases mediated by lymphocytes, in a subject in need of such treatment, which method comprises administering to said subject an effective amount of a compound of formula I or a pharmaceutically acceptable salt thereof.

14. A method for preventing or treating acute or chronic transplant rejection or T-cell mediated inflammatory or autoimmune diseases, in a subject in need of such treatment, which method comprises administering to said subject an effective amount of a compound of formula I or a pharmaceutically acceptable salt thereof.

15. (canceled)
